SEQUENCE LISTING (31 sequences)

SEQ ID NO: 1 - protein, 307 aa, Homo sapiens
MGGFQRGKYGTMAEGRSEDNLSATPPALRIILVGKTGCGKSATGNSILGQPVFESKLRAQSVTRTCQVKTGTWNGRKVLVVDTPSIFESQADTQELYKNIGDCYLLSAPGPHVLLLVIQLGRFTAQDTVAIRKVKEVFGTGAMRHVVILFTHKEDLGGQALDDYVANTDNCSLKDLVRECERRYCAFNNWGSVEEQRQQQAELLAVIERLGREREGSFHSNDLFLDAQLLQRTGAGACQEDYRQYQAKVEWQVEKHKQELRENESNWAYKALLRVKHLMLLHYEIFVFLLLCSILFFIIFLFIFHYI

SEQ ID NO: 2 - protein, 442 aa, Homo sapiens
MYQSLAMAANHGPPPGAYEAGGPGAFMHGAGAASSPVYVPTPRVPSSVLGLSYLQGGGAGSASGGASGGSSGGAASGAGPGTQQGSPGWSQAGADGAAYTPPPVSPRFSFPGTTGSLAAAAAAAAAREAAAYSSGGGAAGAGLAGREQYGRAGFAGSYSSPYPAYMADVGASWAAAAAASAGPFDSPVLHSLPGRANPAARHPNLDMFDDFSEGRECVNCGAMSTPLWRRDGTGHYLCNACGLYHKMNGINRPLIKPQRRLSASRRVGLSCANCQTTTTTLWRRNAEGEPVCNACGLYMKLHGVPRPLAMRKEGIQTRKRKPKNLNKSKTPAAPSGSESLPPASGASSNSSNATTSSSEEMRPIKTEPGLSSHYGHSSSVSQTFSVSAMSGHGPSIHPVLSALKLSPQGYASPVSQSPQTSSKQDSWNSLVLADSHGDIITA

SEQ ID NO: 3 - protein, 373 aa, Homo sapiens
MASELAMSNSDLPTSPLAMEYVNDFDLMKFEVKKEPVETDRIISQCGRLIAGGSLSSTPMSTPCSSVPPSPSFSAPSPGSGSEQKAHLEDYYWMTGYPQQLNPEALGFSPEDAVEALISNSHQLQGGFDGYARGAQQLAAAAGAGAGASLGGSGEEMGPAAAVVSAVIAAAAAQSGAGPHYHHHHHHAAGHHHHPTAGAPGAAGSAAASAGGAGGAGGGGPASAGGGGGGGGGGGGGGAAGAGGALHPHHAAGGLHFDDRFSDEQLVTMSVRELNRQLRGVSKEEVIRLKQKRRTLKNRGYAQSCRFKRVQQRHVLESEKNQLLQQVDHLKQEISRLVRERDAYKEKYEKLVSSGFRENGSSSDNPSSPEFFM

SEQ ID NO: 4 - protein, 477 aa, Homo sapiens
MAQRYDELPHYGGMDGVGVPASMYGDPHAPRPIPPVHHLNHGPPLHATQHYGAHAPHPNVMPASMGSAVNDALKRDKDAIYGHPLFPLLALVFEKCELATCTPREPGVAGGDVCSSDSFNEDIAVFAKQVRAEKPLFSSNPELDNLMIQAIQVLRFHLLELEKVHELCDNFCHRYISCLKGKMPIDLVIDERDGSSKSDHEELSGSSTNLADHNPSSWRDHDDATSTHSAGTPGPSSGGHASQSGDNSSEQGDGLDNSVASPGTGDDDDPDKDKKRQKKRGIFPKVATNIMRAWLFQHLTHPYPSEEQKKQLAQDTGLTILQVNNWFINARRRIVQPMIDQSNRAGFLLDPSVSQGAAYSPEGQPMGSFVLDGQQHMGIRPAGLQSMPGDYVSQGGPMGMSMAQPSYTPPQMTPHPTQLRHGPPMHSYLPSHPHHPAMMMHGGPPTHPGMTMSAQSPTMLNSVDPNVGGQVMDIHAQ

SEQ ID NO: 5 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 6 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 7 - protein, 77 aa, Homo sapiens
MNLRLCVQALLLLWLSLTAVCGGSLMPLPDGNGLEDGNVRHLVQPRGSRNGPGPWQGGRRKFRRQRPRLSHKGPMPF

SEQ ID NO: 8 - protein, 439 aa, Homo sapiens
MPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 9 - protein, 595 aa, Homo sapiens
MALTDGGWCLPKRFGAAGADASDSRAFPAREPSTPPSPISSSSSSCSRGGERGPGGASNCGTPQLDTEAAAGPPARSLLLSSYASHPFGAPHGPSAPGVAGPGGNLSSWEDLLLFTDLDQAATASKLLWSSRGAKLSPFAPEQPEEMYQTLAALSSQGPAAYDGAPGGFVHSAAAAAAAAAAASSPVYVPTTRVGSMLPGLPYHLQGSGSGPANHAGGAGAHPGWPQASADSPPYGSGGGAAGGGAAGPGGAGSAAAHVSARFPYSPSPPMANGAAREPGGYAAAGSGGAGGVSGGGSSLAAMGGREPQYSSLSAARPLNGTYHHHHHHHHHHPSPYSPYVGAPLTPAWPAGPFETPVLHSLQSRAGAPLPVPRGPSADLLEDLSESRECVNCGSIQTPLWRRDGTGHYLCNACGLYSKMNGLSRPLIKPQKRVPSSRRLGLSCANCHTTTTTLWRRNAEGEPVCNACGLYMKLHGVPRPLAMKKEGIQTRKRKPKNINKSKTCSGNSNNSIPMTPTSTSSNSDDCSKNTSPTTQPTASGAGAPVMTGAGESTNPENSELKYSGQDGLYIGVSLASPAEVTSSVRPDSWCALALA

SEQ ID NO: 10 - DNA, 23 bp, synthetic construct
  source 1..23: mol_type = other DNA
aagataacttgtctgcaacacca

SEQ ID NO: 11 - DNA, 20 bp, synthetic construct
  source 1..20: mol_type = other DNA
gtagcagtccccgatgttct

SEQ ID NO: 12 - DNA, 20 bp, synthetic construct
  source 1..20: mol_type = other DNA
ggggaggacgttcatagctt

SEQ ID NO: 13 - DNA, 19 bp, synthetic construct
  source 1..19: mol_type = other DNA
tgctccagggtccagagat

SEQ ID NO: 14 - DNA, 21 bp, synthetic construct
  source 1..21: mol_type = other DNA
gataacttgtctgcaacacca

SEQ ID NO: 15 - DNA, 20 bp, synthetic construct
  source 1..20: mol_type = other DNA
tgtcctgagcagtgaaacgc

SEQ ID NO: 16 - DNA, 21 bp, synthetic construct
  source 1..21: mol_type = other DNA
acatggaacgggaggaaagtc

SEQ ID NO: 17 - DNA, 23 bp, synthetic construct
  source 1..23: mol_type = other DNA
cacgaggatctgagtctgaattg

SEQ ID NO: 18 - protein, 58 aa, Homo sapiens
SATGNSILGQPVFCYLLSAPGPHVLLQQQAELLAVIERLGREREGSFHSNDLFLDAQL

SEQ ID NO: 19 - protein, 58 aa, Pan troglodytes; identical to SEQ ID NO: 18

SEQ ID NO: 20 - protein, 58 aa, unidentified
SATGNSILGQRMFCYLLSAPGPHVLLQQQAELLAVIERLGREREGSFHSNDLFLDAQL

SEQ ID NO: 21 - protein, 58 aa, Rattus norvegicus
SATGNSILRRPAFCYLMCAPGPHVLLGQLAELMALVRRLEQEHEGSFHSNDLFVYTQV

SEQ ID NO: 22 - protein, 58 aa, Mus musculus
SATGNSILRRPAFCYLLCAPGPHVLLGQLAELMALVRRLEQECEGSFHSNDLFLHAEA

SEQ ID NO: 23 - protein, 58 aa, Bos taurus
SATGNSILCQPVFCYLLSVPGPHVLLEQLAQLMAVIEGLEREHQSAFLTNELFFDAQM

SEQ ID NO: 24 - protein, 58 aa, Gallus gallus
SATGNSILGRCAFCVRLSFPGPHALLQQVQQLMEKVVQMVQENEGKYYSNEMYLDPGL

SEQ ID NO: 25 - protein, 58 aa, Homo sp.; identical to SEQ ID NO: 18

SEQ ID NO: 26 - protein, 58 aa, Homo sapiens
SATGNSILGQRRFCYLLSAPGPHALLAQVEQLLGMVEGLVLEHKGAHYSNEVYELAQV

SEQ ID NO: 27 - protein, 58 aa, Homo sapiens
SAAGNSILRKQAFCYLLSAPGPHVLLDQVKELMDCIEDLLMEKNGDHYTNGLYSLIQR

SEQ ID NO: 28 - protein, 58 aa, Homo sapiens
SATGNSILGRKVFCILLTSPGPHALLAQRAQLLGLIQRVVRENKEGCYTNRMYQRAEE

SEQ ID NO: 29 - protein, 59 aa, Homo sapiens
SATGNSILGRDVFAIVLSAPGPHAVLAQLRELMEKVEAIMWENEGDYYSNKYAYQYTQQ

SEQ ID NO: 30 - protein, 58 aa, Homo sapiens
SATANTILGEEIFCIISSCPGPHAIVSQVQELVELIEKMVQCNEGAYFSDDIYKDTEE

SEQ ID NO: 31 - protein, 58 aa, Homo sapiens
SATGNSILGSLVFCLSCCEKGDTFFVTQVKALLTKVNDLRKESGWSGYPHTQENVSKL

(19) United States
(12) Patent Application Publication
(10) Pub. No.: US 2025/0345462 A1
(43) Pub. Date: Nov. 13, 2025

(54) COMPOSITIONS AND METHODS FOR TREATING LIVER DISEASE

(71) Applicant: Yale University, New Haven, CT (US)

(72) Inventor: Silvia Vilarinho, New Haven, CT (US)

(21) Appl. No.: 18/867,264

(22) PCT Filed: May 22, 2023
(86) PCT No.: PCT/US2023/023121

§ 371 Date: Nov. 19, 2024

Related U.S. Application Data
(60) Provisional application No. 63/365,138, filed on May 23, 2022.

(51) Int. Cl. A61K 48/00; A61K 31/7105; A61P 1/16; C12N 15/11; C12N 15/864

(52) U.S. Cl. CPC A61K 48/005; A61K 31/7105; A61K 48/0041; A61P 1/16; C12N 15/11; C12N 15/8645; C12N 2750/14143

(57) ABSTRACT

The present disclosure relates, in part, to a composition comprising a nucleic acid, wherein the nucleic acid encodes a protein which has a reduced abundance in a GTPase IMAP family 5 (GIMAP5) deficient subject or at least partially inhibits expression of a protein which has an increased abundance in a GIMAP5 deficient subject, as compared to a healthy subject. In certain embodiments, the composition is a nucleic acid-lipid particle. In certain embodiments, the composition is a polymer-based vehicle. In another aspect, the present disclosure relates to a recombinant viral vector comprising a nucleic acid encoding a protein which has a reduced abundance in a GIMAP5 deficient subject as compared to a healthy subject. In yet another aspect, the present disclosure provides a method of treating, ameliorating, and/or preventing liver disease and/or portal hypertension in a subject with administration of one or more compositions and/or vectors of the present disclosure.

### CROSS-REFERENCE TO RELATED APPLICATIONS

This application claims priority under 35 U.S.C. § 119 (e) to U.S. Provisional Patent Application No. 63/365,138, filed May 23, 2022, which is hereby incorporated herein by reference in its entirety.

### STATEMENT REGARDING FEDERALLY SPONSORED RESEARCH OR DEVELOPMENT

This invention was made with government support under K08DK113109 and P30DK034989 awarded by the National Institutes of Health. The government has certain rights in the invention.

### SEQUENCE LISTING

The Extensible Markup Language (XML) file named “047162-7375WO1 Sequence Listing.xml” created on May 4, 2023, comprising 31.5 Kbytes, is hereby incorporated herein by reference in its entirety.

### BACKGROUND OF THE INVENTION

Despite major advances in the diagnosis and treatment of viral causes of hepatitis, the incidence of chronic liver disease continues to rise worldwide, affecting up to 1.5 billion people globally and leading to approximately 2 million deaths annually. Because the demand for liver transplantation far exceeds the supply of available donor organs, understanding the pathogenesis of advanced liver disease and its complications is required to develop new therapies to reduce adverse disease outcomes. Portal hypertension, which comprises increased hepatic resistance to blood flow entering the liver, is a major contributor to the morbidity and mortality of liver disease owing to development of ascites, esophageal varices, hemorrhage, and hepatic encephalopathy. While portal hypertension is commonly regarded as a simple consequence of liver damage, phenotypic changes in hepatic endothelial cells can contribute to portal hypertension.

Under physiological conditions, liver sinusoidal endothelial cells (LSECs), representing the major liver endothelial cell subpopulation, contain fenestrae (i.e., non-diaphragmed pores), lack a basement membrane, and do not express CD34. Preceding liver fibrosis, LSECs undergo a capillarization process that results in loss of fenestration and the development of an organized basement membrane, both of which contribute to portal hypertension. These changes are marked by increased expression of CD34. Further, the pathophysiology and possible genetic basis of these changes remain unknown.

Thus, there is a need in the art for compositions and/or methods for treating, preventing, and/or ameliorating liver disease and/or portal hypertension in a subject in need thereof. The present disclosure addresses this need.

### BRIEF DESCRIPTION OF THE FIGURES

The drawings illustrate generally, by way of example, but not by way of limitation, various embodiments of the present application.

FIGS. 1A-1B provides a pedigree showing recessive LOF mutations in GIMAP5 identified in four kindreds. FIG. 1A: Pedigrees depict four unrelated families; affected and unaffected subjects are shown as black-filled and white-filled symbols, respectively; mutations (p.I47T in kindred 1, p.L223P in kindred 2, p.P109L in kindred 3, and p.L204P in kindred 4) are homozygous in the available affected subjects and heterozygous in the parents; consanguineous unions are denoted by a doubleline. FIG. 1B: Schematic representation of human GIMAP5 protein with AIG1 domain depicted, and wherein all four missense mutations are located; conservation of Ile47, Pro109, Leu204, and Leu223 positions across orthologues and GIMAP family members are shown; amino acid positions identical to the human reference are indicated.

FIGS. 2A-2B show liver histology and CD34 expression in humans and mice with GIMAP5 deficiency. FIG. 2A: Photomicrographs of abnormal CD34 immunostaining in liver sinusoids of four unrelated patients (P1-1, P2-1, P3-1, and P4-1) as compared with an unaffected control. FIG. 2B. Photomicrographs of abnormal CD34 sinusoidal expression is shown in the liver sections from C57BL/6 Gimap5sph/sph mice at 2, 3, and >7 week old and in adult C57BL/6 Gimap5sph/sphRag1−/−mice as compared with Gimap5sph/+ control mice. Histological data from 2-week-old, 3-week-old, adult Gimap5sph/sph, and adult Gimap5sph/sphRag1−/−mice was verified at least four, two, seven, and three independent times, respectively. Littermates were used as controls. Scale bar=50 μm.

FIGS. 3A-3F show that genetic deficiency of GIMAP5 causes liver endothelial abnormalities. FIG. 3A: Gimap5 mRNA expression in sorted liver endothelial cells (LECs; DAPI-CD45-CD31+) from C57BL/6 WT and Rag1−/−mice, and splenocytes, sorted Kupffer cells (DAPI-CD45+CD115+F4/80+), and hepatocytes from C57BL/6 mice. FIG. 3B: Immunoblot for GIMAP5 in Gimap5sph/+ and Gimap5sph/sph splenocytes and hepatocytes and in sorted LECs and liver CD45+ cells from Gimap5sph/+ mice. GAPDH is shown as a loading control. FIG. 3C: Confocal microscopy of LECs isolated from Gimap5sph/+ and Gimap5sph/sph and stained for GIMAP5, Lamp-1, and cytochrome-c, and counterstained with DAPI. Scale bars=5 μm. FIG. 3D: LECs isolated from Gimap5sph/+, Gimap5sph/sph, and Gimap5sph/sphRag1−/−livers (left panels) and respective Ly6a and CD34 surface expression (right panels). FIG. 3E: Absolute number of LECs that do or do not express CD34 in Gimap5sph/+ (n=6) and Gimap5sph/sph (n=5) livers. FIG. 3F: Histological and flow cytometric analysis of sinusoidal CD34 expression in tamoxifen-treated adult Gimap5flx/flxxCdh5 (PAC)-CreERT2 and Gimap5flx/flxxVav1-Cre mice (upper and bottom panels, respectively). Experimental data were verified in at least two independent experiments, and littermates were used as controls. Scale bars=50 μm. Numbers depict the percentage of total cells. Student's two-tailed t test was used. *, P<0.05; **, P<0.005; ***, P<0.0005. Adult mice, >7 week old.

FIGS. 4A-4F show scRNA-seq analysis of liver endothelial cells from Gimap5sph/+ and Gimap5sph/sph mice. FIG. 4A: Clustering of endothelial cells from Gimap5sph/+ and Gimap5sph/sph livers using the Louvain method with a resolution parameter 0.4; Uniform Manifold Approximation and Projection (UMAP); cell identities were inferred from marker genes. FIG. 4B: Absolute number of cells in each endothelial subpopulation. FIG. 4C: Overlay of endothelial cell cluster maps annotated by genotype: Gimap5sph/+ and Gimap5sph/sph. FIG. 4D: Pecam1, Clec4g, Dnase311, Ly6a, Cd34, and Gata4 expression in combined subpopulations of LSECs and CECs isolated from Gimap5sph/+ and Gimap5sph/sph mice. FIG. 4E: Enrichment plot from GSEA of pre-ranked list of genes differentially expressed in GIMAP5-deficient and sufficient liver endothelial cells as compared with a background list of mouse Gata4-dependent liver endothelial cell regulated genes. FIG. 4F: Cluster of LSECs, CECs, and macrovascular-like cells with trajectory analysis performed in Monocle.

FIGS. 5A-5F. Representative images of histological findings seen in liver biopsies from patients (P1-1, P2-1, P3-1, and P4-1) with biallelic mutations in GIMAP5. FIG. 5A: P1-1's liver biopsy shows lobular parenchyma devoid of any steatosis, hepatocytic ballooning, or acidophil necrosis; minimal and focal lymphocytic infiltrates (arrow) are seen along with dilated channels at the periphery of the portal tracts, with some of them extending into the sinusoids (left panel; H&E stain); middle panel reveals nodular architecture due to areas of regeneration consisting of thickened hepatic cell plates next to areas of atrophy without any intervening fibrosis (reticulin stain); right panel shows few thin septa in the left aspect of the picture (arrow) but lack of cirrhosis (trichrome stain). FIG. 5B: P2-1's liver biopsy shows hepatocytes with occasional foci of lobular inflammation (arrow; left panel; H&E stain); middle panel shows liver nodularity with zones of widened two-cell-thick plates consistent with hepatic regeneration bounded by narrow compressed liver cell plates (reticulin stain); right panel shows periportal fibrosis (arrow; trichrome stain). FIG. 5C: P3-1's liver biopsy shows no significant abnormalities in the hepatocytes (left panel; H&E stain); middle panel shows areas of regeneration consisting of thickened hepatic cell plates (reticulin stain); right panel shows an area of hepatocyte loss with parenchymal collapse and fibrosis (arrow) but no evidence of cirrhosis (trichrome stain). FIG. 5D: P4-1's liver biopsy shows a portal tract with minimal and focal lymphocytic infiltrates (arrow); the lobular parenchyma is devoid of any steatosis, hepatocytic ballooning, or acidophil necrosis (left panel; H&E stain); middle panel highlights an area of pericentral hepatocyte loss with parenchymal collapse and thin fibrous septa. The liver shows vague nodularity with areas of hepatic regeneration but no cirrhosis (reticulin stain); right panel reveals few thin septa, but lacks features of cirrhosis (trichrome stain). FIG. 5E: Normal-appearing portal tract (oval) and a central venule (asterisk); the portal tract depicts a hepatic arteriole, bile duct, and portal venule; the portal venule is the largest structure with the widest lumen and a thin wall (H&E stain); middle panel shows hepatic cords in a single thick cord plate; the central vein is seen near the center, and a portal tract is at the periphery (reticulin stain); right panel shows normal architecture of the liver and no fibrosis (trichrome stain). Scale bars-50 μm.

FIGS. 6A-6C show liver pathology of Gimap5sph/+Rag1−/− and Gimap5sph/sphRag1−/− mice. FIG. 6A: Gross liver morphology of adult Gimap5sph/+Rag1−/−(smooth) and Gimap5sph/sphRag1−/−mice (nodular). FIG. 6B: Reticulin and trichrome stains of liver sections from Gimap5sph/+Rag1−/−(left panels) and Gimap5sph/sphRag1−/−(right panels) show two-cell-thick plate and increased collagen deposition solely in mutant mice. FIG. 6C: Flow cytometric analysis shows increased CD34 expression in liver endothelial cells (DAPI-CD45-CD31+) isolated from Gimap5sph/sphRag1−/−mice as compared with Gimap5sph/+Rag1−/−mice. Experimental data were verified in at least two independent experiments, and littermates were used as controls. Scale bars=50 μm.

FIGS. 7A-7C show subcellular localization of Gimap5 in sorted liver endothelial cells (LECs; DAPI-CD45-CD31+). Confocal microscopy of isolated LECs from Gimap5sph/+ (FIGS. 7A-7B) and Gimap5sph/sph (FIG. 7C) and stained for GIMAP5, lysosomal marker Lamp-1, and mitochondrial marker cytochrome-c, and counterstained with DAPI. Experimental data were verified in at least two independent experiments. Scale bars=5 μm.

### BRIEF SUMMARY OF THE INVENTION

The present disclosure provides a composition comprising one or more nucleic acid-lipid particles, wherein each nucleic acid-lipid particle comprises:
(a) a cationic lipid;
(b) a non-cationic lipid;
(c) a conjugated lipid that inhibits aggregation of two or more nucleic acid lipid particles; and
(d) a nucleic acid encapsulated within the nucleic acid-lipid particle, wherein the nucleic acid either:
   (i) encodes a protein which has a reduced abundance in a GTPase IMAP family member 5 (GIMAP5) deficient subject as compared to a healthy subject; or
   (ii) at least partially inhibits expression of a protein which has an increased abundance in a GIMAP5 deficient subject as compared to a healthy subject.

The present disclosure further provides a composition comprising one or more polymer-based vehicles, wherein the polymer-based vehicle comprises a nucleic acid which is at least partially encapsulated within the polymer-based vehicle, wherein the nucleic acid either:
(a) encodes a protein which has a reduced abundance in a GTPase IMAP family member 5 (GIMAP5) deficient subject as compared to a healthy subject; or
(b) at least partially inhibits expression of a protein which has an increased abundance in a GIMAP5 deficient subject as compared to a healthy subject.

The present disclosure further provides a recombinant viral vector, the vector comprising:
(a) an expression cassette comprising a nucleic acid sequence encoding a protein which has a reduced abundance in a GTPase IMAP family member 5 (GIMAP5) deficient subject as compared to a healthy subject; and
(b) an expression control sequence operably linked to the nucleic acid.

The present disclosure further provides a pharmaceutical composition comprising the composition of the present disclosure or the recombinant viral vector of the present disclosure and a pharmaceutically acceptable carrier.

The present disclosure further provides a method of treating, ameliorating and/or preventing liver disease and/or portal hypertension in a subject, the method comprising administering to the subject in need thereof a therapeutically effective amount of the composition of the present disclosure.

The present disclosure further provides a method of treating, ameliorating and/or preventing liver disease and/or portal hypertension in a subject, the method comprising administering to the subject in need thereof a therapeutically effective amount of the recombinant viral vector of the present disclosure.

The present disclosure further provides a method of treating, ameliorating and/or preventing liver disease and/or portal hypertension in a subject, the method comprising administering to the subject in need thereof a therapeutically effective amount of the pharmaceutical composition of the present disclosure.

### DETAILED DESCRIPTION OF THE INVENTION

Portal hypertension is a major contributor to decompensation and death from liver disease, a global health problem. The present application demonstrates the presence of homozygous damaging mutations in GIMAP5, a small organellar GTPase, in four families with unexplained portal hypertension. It is further shown that GIMAP5 is expressed in hepatic endothelial cells and that its loss in both humans and mice results in capillarization of liver sinusoidal endothelial cells (LSECs), which is also observed when GIMAP5 is selectively deleted in endothelial cells. Single-cell RNA-sequencing analysis in a GIMAP5-deficient mouse model revealed replacement of LSECs with capillarized endothelial cells, a reduction of macrovascular hepatic endothelial cells, and an expansion in lymphatic endothelial cells. Further, the results of this analysis suggested that GIMAP5 might be upstream of GATA4, a transcription factor required for LSEC specification. Thus, the present application demonstrates that GIMAP5 is a critical regulator of liver endothelial cell homeostasis and, when absent, produces portal hypertension. These findings provide new insight into the pathogenesis of portal hypertension, a major contributor to morbidity and mortality from liver disease.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

As used herein, the term “active ingredient” refers to a therapeutic agent that is to be delivered to a subject to produce a therapeutic effect in the subject.

By “aqueous media” is meant water or water containing buffer or salt.

As used herein, the term “aqueous solution” or “aqueous media” refers to a composition comprising in whole, or in part, water.

The term “amphipathic lipid” refers, in part, to any suitable material wherein the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

Representative examples of phospholipids include, but are not limited to, phosphatidylcholine, phosphatidylethanolamine, phosphatidylserine, phosphatidylinositol, phospha-tidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and acyloxyacids, are also within the group desig-nated as amphipathic lipids. Additionally, the amphipathic lipids described above can be mixed with other lipids including triglycerides and sterols.

The term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, C18 alkyl chains, ether linkages between the head group and alkyl chains, and 0 to 3 double bonds. Such lipids include, e.g., DSDMA, DLinDMA, DLenDMA, and DODMA.

In one aspect, the terms “co-administered” and “co-administration” as relating to a subject refer to administering to the subject a compound/composition of the present disclosure or salt thereof along with a compound/composition that may also treat, ameliorate, and/or prevent any disease or disorder contemplated herein and/or with a compound that is useful in treating, ameliorating, and/or preventing other medical conditions but which in themselves may cause or facilitate any disease or disorder contemplated herein. In certain embodiments, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The term “distal site,” as used herein, refers to a physically separated site, which is not limited to an adjacent capillary bed, but includes sites broadly distributed throughout an organism

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

As used herein, “expression cassette” refers to a nucleic acid molecule encoding a gene product of interest, a promoter, and other regulatory sequences for it, wherein the cassette is a viral vector (e.g., a viral particle). In certain embodiments, the expression cassette is packaged within a capsid (i.e., viral vector). Usually, such expression cassettes for making viral vectors are adjacent to the packaging signals of the viral genome and other expression control sequences. For example, in the case of AAV viral vectors, the packaging signals are 5-′inverted terminal repeats (ITR) and 3′-ITR.

The term “encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The term “fusogenic” refers to the ability of a lipid particle, to fuse with the membranes of a cell. The membranes can be either the plasma membrane or membranes surrounding organelles, e.g., endosome, nucleus, etc.

The term “gene” refers to a nucleic acid (e.g., DNA or RNA) sequence that comprises partial length or entire length coding sequences necessary for the production of a polypeptide or precursor polypeptide.

The term “gene product,” as used herein, refers to a product of a gene such as a RNA transcript or a polypeptide.

The term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacyl glycerol, dialkyl glycerol, N-N-dialkylamino, 1,2-diacyloxy-3-aminopropane, and 1,2-dialkyl-3-aminopropane.

When used herein to describe the ratio of lipid: mRNA, the term “lipid” refers to the total lipid in the particle.

As used herein, the term “GIMAP5” refers to GTPase IMAP family member 5, the protein having SEQ ID NO: 1 for the human homolog or the gene encoding that protein.

The term “GIMAP5 promoting agent” refers to any agent which promotes the expression of GIMAP5 in a subject. In various embodiments, the GIMAP5 promoting agent is a nucleic acid encoding GIMAP5. In various embodiments, the GIMAP5 promoting agent is formulated in a lipid formulation. In various embodiments, the GIMAP5 promoting agent is encapsulated in a viral vector.

In general, when referring to “identity,” “homology,” or “similarity” between two different sequences, “identity,” “homology,” or “similarity” is that of an “aligned” sequence. Determined in relation to. An “aligned” sequence or “alignment” refers to a plurality of nucleic acid or protein (amino acid) sequences that often contain corrections for missing or additional bases or amino acids compared to the reference sequence.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid conjugate” refers to a conjugated lipid that inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (see, e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a messenger RNA), with full encapsulation, partial encapsulation, or both. In a preferred embodiment, the nucleic acid is fully encapsulated in the lipid particle (e.g., to form an SPLP, PSPLP, SNALP, or other nucleic acid-lipid particle).

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like. In certain embodiments, the target site within an organism is the liver. In certain embodiments, the active agent or therapeutic (e.g., messenger RNA) is delivered to one or more liver endothelial cells. In certain embodiments, the liver endothelial cell is a liver sinusoidal endothelial cell, liver macrovascular endothelial cell, or a liver lymphatic endothelial cell.

The term “mammal” refers to any mammalian species such as a human, mouse, rat, dog, cat, hamster, guinea pig, rabbit, livestock, and the like.

As the term is used herein, “to modulate” or “modulation of” a biological or chemical process or state refers to the alteration of the normal course of the biological or chemical process, or changing the state of the biological or chemical process to a new state that is different than the present state. For example, modulation of the isoelectric point of a polypeptide may involve a change that increases the isoelectric point of the polypeptide. Alternatively, modulation of the isoelectric point of a polypeptide may involve a change that decreases the isoelectric point of a polypeptide.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (PI, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, IRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector. “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

These control sequences are “operably linked” coding sequence. As used herein, the term “operably linked” refers to an expression control sequence that is close to a gene of interest and an expression control that acts trans or distantly to control the gene of interest. Refers to both with an array.

As used herein, the term “pharmaceutical composition” or “composition” refers to a mixture of at least one composition or recombinant viral vector useful within the present disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the present disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the subject. Supplementary active compounds may also be incorporated into the compositions. The “pharmaceutically acceptable carrier” may further include a pharmaceutically acceptable salt of the compound useful within the present disclosure. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The terms “polynucleotide” and “oligonucleotide” as used herein, refer to a polymer or oligomer of nucleotide or nucleoside monomers comprising naturally occurring bases, sugars and intersugar (backbone) linkages. The terms “polynucleotide” and “oligonucleotide” also include polymers or oligomers comprising non-naturally occurring monomers, or portions thereof, which function similarly. Such modified or substituted oligonucleotides are often preferred over native forms because of properties such as, for example, enhanced cellular uptake, reduced immunogenicity, and increased stability in the presence of nucleases. Oligonucleotides are generally classified as deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribooligonucleotide consists of a similar repeating structure where the 5-carbon sugar is ribose.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “reduced abundance” as used herein refers to a decreased relative functional amount of a given protein in one subject and/or cell as compared to another. In certain embodiments, the protein may be present in approximately equivalent amounts in two subjects and/or cells, but the protein in a first subject is partially functional or non-functional (i.e., comprises mutations, processing errors, and/or is misfolded) as compared to a second subject, thereby effectively representing a reduced abundance in the first subject. The term “increased abundance” as used herein refers to an increased relative functional amount of a given protein in one subject and/or cell as compared to another.

The term “salt” includes any anionic and cationic complex, such as the complex formed between a cationic lipid and one or more anions. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkyl sulfonate, an aryl sulfonate, arsenate, arsenite, chromate, dichromate, cyanide, cyanate, thiocyanate, hydroxide, peroxide, permanganate, and mixtures thereof. In particular embodiments, the salts of the cationic lipids disclosed herein are crystalline salts.

The terms “sequence homology,” “percent identity (%),” “sequence identity,” “sequence identity percent,” or “percent identity” in the context of nucleic acid and/or amino acid sequences refers to a quantitative measurement of the similarity between two nucleic acid or amino acid sequences (e.g., DNA, amino acid or otherwise).

The term “serum-stable” as used herein in relation to nucleic acid-lipid particles means that the particle is not significantly degraded after exposure to a serum or nuclease assay that would significantly degrade free DNA or RNA. Suitable assays include, for example, a standard serum assay, a DNAse assay, or an RNAse assay.

The term “SNALP” as used herein, refers to a stable nucleic acid-lipid particle, which term may be used interchangeably with nucleic acid-lipid particle. A SNALP represents a particle made from lipids (e.g., a cationic lipid, a non-cationic lipid, and a conjugated lipid that prevents aggregation of the particle), wherein the nucleic acid (e.g., mRNA, siRNA, aiRNA, miRNA, ssDNA, dsDNA, ssRNA, short hairpin RNA (shRNA), dsRNA, or a plasmid, including plasmids from which an interfering RNA is transcribed) is fully encapsulated within the lipid. As used herein, the term “SNALP” includes an SPLP, which is the term used to refer to a nucleic acid-lipid particle comprising a nucleic acid (e.g., a plasmid) encapsulated within the lipid. SNALP and SPLP typically contain a cationic lipid, a non-cationic lipid, and a lipid conjugate (e.g., a PEG-lipid conjugate). SNALP and SPLP are useful for systemic applications, as they can exhibit extended circulation lifetimes following intravenous (i.v.) injection, they can accumulate at distal sites (e.g., sites physically separated from the administration site), and they can mediate expression of the transfected gene or silencing of target gene expression at these distal sites. SPLP include “pSPLP,” which comprise an encapsulated condensing agent-nucleic acid complex as set forth in PCT Publication No. WO 2000/03683, the disclosure of which is herein incorporated by reference in its entirety for all purposes.

The nucleic acid-lipid particles of the present disclosure typically have a mean diameter of from about 40 nm to about 150 nm, from about 50 nm to about 150 nm, from about 60 nm to about 130 nm, from about 70 nm to about 110 nm, or from about 70 to about 90 nm, and are substantially non-toxic. In addition, nucleic acids, when present in the lipid particles of the disclosure, are resistant in aqueous solution to degradation with a nuclease. Nucleic acid-lipid particles and their method of preparation are disclosed in, e.g., U.S. Patent Publication Nos. 20040142025 and 20070042031, the disclosures of which are herein incorporated by reference in their entirety for all purposes.

As used herein, a “subject” may be a human or non-human mammal or a bird. Non-human mammals include, for example, livestock and pets, such as ovine, bovine, porcine, canine, feline and murine mammals. In certain embodiments, the subject is human.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0 9, 0.8, 0.7, 0.6, 0.5, 0 4, 0.3, 0.2, 0.1, 0.01, or about 0.001 wt % or less, or about 0 wt %.

The terms “substantially identical” or “substantial identity,” in the context of two or more nucleic acids, refer to two or more sequences or subsequences that are the same or have a specified percentage of nucleotides that are the same (i.e., at least about 60%, in certain embodiments at least about 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. This definition, when the context indicates, also refers analogously to the complement of a sequence. In certain embodiments, the substantial identity exists over a region that is at least about 5, 10, 15, 20, 25, 30, 35, 40, 45, 50, 55, or 60 nucleotides in length.

The term “systemic delivery,” as used herein, refers to delivery of lipid particles that leads to a broad biodistribution of an active agent or therapeutic agent such as an interfering RNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood life-time such that the agent is not rapidly degraded or cleared (e.g., such as by first pass organs, such as the liver and lung, etc., or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

As used herein, the terms “transfect” or “transfection” mean the intracellular introduction of a mRNA into a cell, or preferably into a target cell. The introduced mRNA may be stably or transiently maintained in the target cell. The term “transfection efficiency” refers to the relative amount of mRNA taken up by the target cell which is subject to transfection. In practice, transfection efficiency is estimated by the amount of a reporter nucleic acid product expressed by the target cells following transfection. Preferred embodiments include compositions with high transfection efficacies and in particular those compositions that minimize adverse effects which are mediated by transfection of non-target cells. The compositions of the present disclosure that demonstrate high transfection efficacies improve the likelihood that appropriate dosages of the mRNA will be delivered to the target cell, while minimizing potential systemic adverse effects. In one embodiment of the present disclosure, the lipid particles of the present disclosure are capable of delivering large mRNA sequences (e.g., mRNA of at least 1 kDa, 1.5 kDa, 2 kDa, 2.5 kDa, 5 kDa, 10 kDa, 12 kDa, 15 kDa, 20 kDa, 25 kDa, 30 kDa, or more).

The term “treat,” “treating” or “treatment,” as used herein, means reducing the frequency or severity with which symptoms of a disease or condition are experienced by a subject by virtue of administering an agent or compound to the subject.

The term “vehicle” as used herein refer to a carrier and/or inert medium in which an active agent (e.g., nucleic acid) is formulated and/or administered.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

Certain abbreviations used herein include: AIG1, GTP-binding AIG1 homology domain; TD, transmembrane domain; mut, mutant; wt, wild-type; LEC, liver endothelial cells; LSEC, liver sinusoidal endothelial cells; APC, allophycocyanin; Cy, cyanine; LOF, loss of function; CEC, capillarized endothelial cells; GSEA, gene set enrichment analysis; and i.p., intraperitoneally.

### Nucleic Acid-Lipid Particles

In one aspect, the present disclosure provides a composition comprising one or more nucleic acid-lipid particles, wherein each nucleic acid-lipid particle comprises:
(a) a cationic lipid;
(b) a non-cationic lipid;
(c) a conjugated lipid that inhibits aggregation of two or more nucleic acid lipid particles; and
(d) a nucleic acid encapsulated within the nucleic acid-lipid particle, wherein the nucleic acid either:
   (i) encodes a protein which has a reduced abundance in a GTPase IMAP family member 5 (GIMAP5) deficient subject as compared to a healthy subject; or
   (ii) at least partially inhibits expression of a protein which has an increased abundance in a GIMAP5 deficient subject as compared to a healthy subject.

In certain embodiments, the cationic lipid comprises about 50 mol % to about 90 mol % of the total lipid present in the nucleic acid-lipid particle.

In certain embodiments, the non-cationic lipid is at least one selected from the group consisting of cholesterol and a phospholipid.

In certain embodiments, wherein the non-cationic lipid comprises about 9.9 mol % to about 49.9 mol % of the total lipid present in the nucleic acid-lipid particle.

In certain embodiments, the conjugated lipid that inhibits aggregation of two or more nucleic acid-lipid particles comprises a polyethyleneglycol (PEG)-lipid conjugate.

In certain embodiments, the conjugated lipid comprises about 0.1 mol % to about 2 mol % of the total lipid present in the nucleic acid-lipid particle.

In certain embodiments, the nucleic acid-lipid particle is a nanoparticle.

In certain embodiments, the protein which has a reduced abundance in a GTPase IMAP family member 5 (GIMAP5) deficient subject as compared to a healthy subject is an enzyme. In certain embodiments, the enzyme is GIMAP5. In certain embodiments, the nucleic acid comprises a messenger RNA (mRNA) which encodes a protein that shares at least 85% sequence homology with SEQ ID NO:1. In certain embodiments, the nucleic acid comprises a messenger RNA (mRNA) which encodes a protein that shares at least 90% sequence homology with SEQ ID NO: 1. In certain embodiments, the nucleic acid comprises a messenger RNA (mRNA) which encodes a protein that shares at least 95% sequence homology with SEQ ID NO:1. In certain embodiments, the nucleic acid comprises a messenger RNA (mRNA) which encodes a protein that shares at least 96% sequence homology with SEQ ID NO:1. In certain embodiments, the nucleic acid comprises a messenger RNA (mRNA) which encodes a protein that shares at least 97% sequence homology with SEQ ID NO:1. In certain embodiments, the nucleic acid comprises a messenger RNA (mRNA) which encodes a protein that shares at least 98% sequence homology with SEQ ID NO:1. In certain embodiments, the nucleic acid comprises a messenger RNA (mRNA) which encodes a protein that shares at least 99% sequence homology with SEQ ID NO: 1 In certain embodiments, the mRNA encodes SEQ ID NO:1.

In certain embodiments, the protein which has a reduced abundance in a GTPase IMAP family member 5 (GIMAP5) deficient subject as compared to a healthy subject is GATA4. In certain embodiments, the nucleic acid comprises a messenger RNA (mRNA) which encodes a protein that shares at least 85% sequence homology with SEQ ID NO:2. In certain embodiments, the nucleic acid comprises a messenger RNA (mRNA) which encodes a protein that shares at least 90% sequence homology with SEQ ID NO:2. In certain embodiments, the nucleic acid comprises a messenger RNA (mRNA) which encodes a protein that shares at least 95% sequence homology with SEQ ID NO:2. In certain embodiments, the nucleic acid comprises a messenger RNA (mRNA) which encodes a protein that shares at least 96% sequence homology with SEQ ID NO:2. In certain embodiments, the nucleic acid comprises a messenger RNA (mRNA) which encodes a protein that shares at least 97% sequence homology with SEQ ID NO: 2. In certain embodiments, the nucleic acid comprises a messenger RNA (mRNA) which encodes a protein that shares at least 98% sequence homology with SEQ ID NO:2. In certain embodiments, the nucleic acid comprises a messenger RNA (mRNA) which encodes a protein that shares at least 99% sequence homology with SEQ ID NO:2. In certain embodiments, the mRNA encodes SEQ ID NO:2.

In certain embodiments, the protein which has a reduced abundance in a GTPase IMAP family member 5 (GIMAP5) deficient subject as compared to a healthy subject is MAF. In certain embodiments, the nucleic acid comprises a messenger RNA (mRNA) which encodes a protein that shares at least 85% sequence homology with SEQ ID NO:3. In certain embodiments, the nucleic acid comprises a messenger RNA (mRNA) which encodes a protein that shares at least 90% sequence homology with SEQ ID NO:3. In certain embodiments, the nucleic acid comprises a messenger RNA (mRNA) which encodes a protein that shares at least 95% sequence homology with SEQ ID NO:3. In certain embodiments, the nucleic acid comprises a messenger RNA (mRNA) which encodes a protein that shares at least 96% sequence homology with SEQ ID NO:3. In certain embodiments, the nucleic acid comprises a messenger RNA (mRNA) which encodes a protein that shares at least 97% sequence homology with SEQ ID NO: 3. In certain embodiments, the nucleic acid comprises a messenger RNA (mRNA) which encodes a protein that shares at least 98% sequence homology with SEQ ID NO:3. In certain embodiments, the nucleic acid comprises a messenger RNA (mRNA) which encodes a protein that shares at least 99% sequence homology with SEQ ID NO:3. In certain embodiments, the mRNA encodes SEQ ID NO:3.

In certain embodiments, the protein which has a reduced abundance in a GTPase IMAP family member 5 (GIMAP5) deficient subject as compared to a healthy subject is MEIS2. In certain embodiments, the nucleic acid comprises a messenger RNA (mRNA) which encodes a protein that shares at least 85% sequence homology with SEQ ID NO:4. In certain embodiments, the nucleic acid comprises a messenger RNA (mRNA) which encodes a protein that shares at least 90% sequence homology with SEQ ID NO:4. In certain embodiments, the nucleic acid comprises a messenger RNA (mRNA) which encodes a protein that shares at least 95% sequence homology with SEQ ID NO:4. In certain embodiments, the nucleic acid comprises a messenger RNA (mRNA) which encodes a protein that shares at least 96% sequence homology with SEQ ID NO:4. In certain embodiments, the nucleic acid comprises a messenger RNA (mRNA) which encodes a protein that shares at least 97% sequence homology with SEQ ID NO: 4. In certain embodiments, the nucleic acid comprises a messenger RNA (mRNA) which encodes a protein that shares at least 98% sequence homology with SEQ ID NO:4. In certain embodiments, the nucleic acid comprises a messenger RNA (mRNA) which encodes a protein that shares at least 99% sequence homology with SEQ ID NO:4. In certain embodiments, the mRNA encodes SEQ ID NO:4.

In certain embodiments, the nucleic acid at least partially inhibits expression of a protein which has an increased abundance in a GIMAP5 deficient subject as compared to a healthy subject.

In certain embodiments, the protein which has an increased abundance in a GIMAP5 deficient subject is PDGFβ. In certain embodiments, the protein which has an increased abundance in a GIMAP5 deficient subject is VEGFα. In certain embodiments, the protein which has an increased abundance in a GIMAP5 deficient subject is APLN. In certain embodiments, the protein which has an increased abundance in a GIMAP5 deficient subject is MYC. In certain embodiments, the protein which has an increased abundance in a GIMAP5 deficient subject is GATA6.

In certain embodiments, the nucleic acid comprises a small interfering RNA (siRNA).

In certain embodiments, the siRNA at least partially inhibits expression of a protein that shares at least 85% sequence homology with SEQ ID NO:5. In certain embodiments, the siRNA at least partially inhibits expression of a protein that shares at least 90% sequence homology with SEQ ID NO:5. In certain embodiments, the siRNA at least partially inhibits expression of a protein that shares at least 95% sequence homology with SEQ ID NO:5. In certain embodiments, the siRNA at least partially inhibits expression of a protein that shares at least 96% sequence homology with SEQ ID NO:5. In certain embodiments, the siRNA at least partially inhibits expression of a protein that shares at least 97% sequence homology with SEQ ID NO: 5. In certain embodiments, the siRNA at least partially inhibits expression of a protein that shares at least 98% sequence homology with SEQ ID NO:5. In certain embodiments, the siRNA at least partially inhibits expression of a protein that shares at least 99% sequence homology with SEQ ID NO:5. In certain embodiments, the siRNA at least partially inhibits expression of a protein with sequence SEQ ID NO:5.

In certain embodiments, the siRNA at least partially inhibits expression of a protein that shares at least 85% sequence homology with SEQ ID NO:6. In certain embodiments, the siRNA at least partially inhibits expression of a protein that shares at least 90% sequence homology with SEQ ID NO:6. In certain embodiments, the siRNA at least partially inhibits expression of a protein that shares at least 95% sequence homology with SEQ ID NO:6. In certain embodiments, the siRNA at least partially inhibits expression of a protein that shares at least 96% sequence homology with SEQ ID NO:6. In certain embodiments, the siRNA at least partially inhibits expression of a protein that shares at least 97% sequence homology with SEQ ID NO: 6. In certain embodiments, the siRNA at least partially inhibits expression of a protein that shares at least 98% sequence homology with SEQ ID NO:6. In certain embodiments, the siRNA at least partially inhibits expression of a protein that shares at least 99% sequence homology with SEQ ID NO:6. In certain embodiments, the siRNA at least partially inhibits expression of a protein with sequence SEQ ID NO:6.

In certain embodiments, the siRNA at least partially inhibits expression of a protein that shares at least 85% sequence homology with SEQ ID NO:7. In certain embodiments, the siRNA at least partially inhibits expression of a protein that shares at least 90% sequence homology with SEQ ID NO:7. In certain embodiments, the siRNA at least partially inhibits expression of a protein that shares at least 95% sequence homology with SEQ ID NO:7. In certain embodiments, the siRNA at least partially inhibits expression of a protein that shares at least 96% sequence homology with SEQ ID NO:7. In certain embodiments, the siRNA at least partially inhibits expression of a protein that shares at least 97% sequence homology with SEQ ID NO: 7. In certain embodiments, the siRNA at least partially inhibits expression of a protein that shares at least 98% sequence homology with SEQ ID NO:7. In certain embodiments, the siRNA at least partially inhibits expression of a protein that shares at least 99% sequence homology with SEQ ID NO:7. In certain embodiments, the siRNA at least partially inhibits expression of a protein with sequence SEQ ID NO:7.

In certain embodiments, the siRNA at least partially inhibits expression of a protein that shares at least 85% sequence homology with SEQ ID NO:8. In certain embodiments, the siRNA at least partially inhibits expression of a protein that shares at least 90% sequence homology with SEQ ID NO:8. In certain embodiments, the siRNA at least partially inhibits expression of a protein that shares at least 95% sequence homology with SEQ ID NO:8. In certain embodiments, the siRNA at least partially inhibits expression of a protein that shares at least 96% sequence homology with SEQ ID NO:8. In certain embodiments, the siRNA at least partially inhibits expression of a protein that shares at least 97% sequence homology with SEQ ID NO: 8. In certain embodiments, the siRNA at least partially inhibits expression of a protein that shares at least 98% sequence homology with SEQ ID NO:8. In certain embodiments, the siRNA at least partially inhibits expression of a protein that shares at least 99% sequence homology with SEQ ID NO:8. In certain embodiments, the siRNA at least partially inhibits expression of a protein with sequence SEQ ID NO:8.

In certain embodiments, the siRNA at least partially inhibits expression of a protein that shares at least 85% sequence homology with SEQ ID NO:9. In certain embodiments, the siRNA at least partially inhibits expression of a protein that shares at least 90% sequence homology with SEQ ID NO:9. In certain embodiments, the siRNA at least partially inhibits expression of a protein that shares at least 95% sequence homology with SEQ ID NO:9. In certain embodiments, the siRNA at least partially inhibits expression of a protein that shares at least 96% sequence homology with SEQ ID NO:9. In certain embodiments, the siRNA at least partially inhibits expression of a protein that shares at least 97% sequence homology with SEQ ID NO: 9. In certain embodiments, the siRNA at least partially inhibits expression of a protein that shares at least 98% sequence homology with SEQ ID NO:9. In certain embodiments, the siRNA at least partially inhibits expression of a protein that shares at least 99% sequence homology with SEQ ID NO:9. In certain embodiments, the siRNA at least partially inhibits expression of a protein with sequence SEQ ID NO:9.

The lipid particles of the present disclosure typically comprise an active agent or therapeutic agent, a cationic lipid, a non-cationic lipid, and a conjugated lipid that inhibits aggregation of particles. In some embodiments, the active agent or therapeutic agent is fully encapsulated within the lipid portion of the lipid particle such that the active agent or therapeutic agent in the lipid particle is resistant in aqueous solution to enzymatic degradation, e.g., by a nuclease or protease. In other embodiments, the lipid particles described herein are substantially non-toxic to mammals such as humans. The lipid particles of the disclosure typically have a mean diameter of from about 40 nm to about 150 nm, from about 50 nm to 10 about 150 nm, from about 60 nm to about 130 nm, from about 70 nm to about 110 nm, or from about 70 to about 90 nm.

In certain embodiments, the nucleic acid-lipid particles of the present disclosure are serum-stable nucleic acid-lipid particles (SNALP) which comprise RNA (e.g., mRNA), a cationic lipid, a non-cationic lipid (e.g., cholesterol alone or mixtures of one or more phospholipids and cholesterol), and a conjugated lipid that inhibits aggregation of the particles (e.g., one or more PEG-lipid conjugates). The SNALP may comprise at least 1, 2, 3, 4, 5, 6, 7, 8, 9, 10, or more unmodified and/or modified mRNA molecules. Nucleic acid-lipid particles and their method of preparation are described in, e.g., U.S. Pat. Nos. 5,753,613; 5,785,992; 5,705,385; 5,976,567; 5,981,501; 6,110,745; 6,320, 25 017; 8,058,069; 8,492,359; 8,822,668; 9,364,435; 9,504,651; and 11,141,378; and PCT Publication No. WO 96/40964, the disclosures of which are each herein incorporated by reference in their entirety for all purposes.

### Cationic Lipids

In the nucleic acid-lipid particles of the disclosure, the cationic lipid may comprise, e.g., one or more of the following: 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-45 dimethy laminopropyl)-[1,3]-dioxolane (D Lin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-Nmethylpepiazino-[1,3]-dioxolane (DLin-K-MPZ), 2,2-dili-noleyl-4-dimethylaminomethyl-[1,3]-dioxolane (DLin-KDMA), 1,2-dilinoleylcarbamoyloxy-3-dimethy laminopropane (D Lin-C-DAP), 1,2-dilinoleyoxy-3-(dimethylaminoacetoxypropane (DLin-DAC), 1-2dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-2-DMAP), 1,2-dilinoleyloxy-3-trimethylaminopropane chloride salt (DLin-TMA.C1), 1,2-dilinoleoyl-3-trimethylaminopropane chloride salt (DLin-TAP.CI), 1,2-dilinoleyloxy-3-(N-methy lpiperazino) propane (D Lin-MPZ), 3-(N,Ndilinoley lamino)-1,2-propanediol (D LinAP), 3-(N,Ndioley lamino)-1,2-propanedio (DOAP), 1,2-dilinoleyloxo-3-(2-N,N-dimethy lamino) ethoxypropane (D Lin-EG-DMA), N,N-dioleyl-N,N-dimethylanrmonium chloride (DODAC), 1,2-dioleyloxy-N,N-dimethylaminopropane (DODMA), 1,2-disteary loxy-N,N-dimethy laminopropane (DSDMA), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N, N-trimethylammonium chloride (DOTAP), 3-(N-(N″,N′dimethylaminoethane)-carbamoyl) cholesterol (DC-Chol), N-(1,2-dimyristyloxyprop-3-yl)-N,N-dimethyl-N-hydroxyethyl anrmonium bromide (DMRIE), 2,3-dioleyloxy-N-[2 (spermine-carboxamidoethyl]-N,N-dimethy 1-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′, 1-2′-octadecadienoxy) propane (CpLinDMA), N,N-dimethyl-3,4-dioleyloxybenzylamine (DMOBA), 1,2-N,N′dioleylcarbamyl-3-dimethylaminopropane (DOcarbDAP), 1,2-N,N′-dilinoleylcarbamyl-3-dimethylaminopropane (DLincarbDAP), or mixtures thereof. In certain embodiments, the cationic lipid is DLinDMA, DLin-K-C2-DMA (“XTC2”), or mixtures thereof.

The synthesis of cationic lipids such as DLin-K-C2-DMA (“XTC2”), DLin-K-C3-DMA, DLin-K-C4-DMA, DLin-K6-DMA, and DLin-K-MPZ, as well as additional cationic lipids, is described in U.S. Provisional Application No. 61/104,212, filed Oct. 9, 2008, the disclosure of which is herein incorporated by reference in its entirety for all purposes. The synthesis of cationic lipids such as DLin-K-DMA, DLin-CDAP, DLin-DAC, DLin-MA, DLinDAP, DLin-S-DMA, DLin-2-DMAP, DLin-TMA.C1, DLin-TAP.CI, DLin-MPZ, DLinAP, DOAP, and DLin-EG-DMA, as well as additional cationic lipids, is described in PCT Application No. PCT/US08/88676, filed Dec. 31, 2008, the disclosure of which is herein incorporated by reference in its entirety for all purposes. The synthesis of cationic lipids such as CLinDMA, as well as additional cationic lipids, is described in U.S. Patent Publication No. 20060240554, the disclosure of which is herein incorporated by reference in its entirety for all purposes.

In some embodiments, the cationic lipid may comprise from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid may comprise from about 55 mol % to about 90 mol %, from about 55 mol % to about 85 mol %, from about 55 mol % to about 80 mol %, from about 55 mol % to about 75 mol %, from about 55 mol % to about 70 mol %, or from about 55 mol % to about 65 mol % of the total lipid present in the particle.

In yet other embodiments, the cationic lipid may comprise from about 60 mol % to about 90 mol %, from about 60 mol % to about 85 mol %, from about 60 mol % to about 80 mol %, from about 60 mol % to about 75 mol %, or from about 60 mol % to about 70 mol % of the total lipid present in the particle.

In still yet other embodiments, the cationic lipid may comprise from about 65 mol % to about 90 mol %, from about 65 mol % to about 85 mol %, from about 65 mol % to about 80 mol %, or from about 65 mol % to about 75 mol % of the total lipid present in the particle.

In further embodiments, the cationic lipid may comprise from about 70 mol % to about 90 mol %, from about 70 mol % to about 85 mol %, from about 70 mol % to about 80 mol %, from about 75 mol % to about 90 mol %, from about 75 mol % to about 85 mol %, or from about 80 mol % to about 90 mol % of the total lipid present in the particle.

In additional embodiments, the cationic lipid may comprise (at least) about 50, 51, 52, 53, 54, 55, 56, 57, 58, 59, 60, 61, 62, 63, 64, 65, 66, 67, 68, 69, 70, 71, 72,73, 74, 75, 76, 77, 78, 79, 80, 81, 82, 83, 84, 85, 86, 87, 88, 89, or 90 mol % (or any fraction thereof or range therein) of the total lipid present in the particle.

### Non-Cationic Lipid

In the nucleic acid-lipid particles of the present disclosure, the non-cationic lipid may comprise, e.g., one or more anionic lipids and/or neutral lipids. In preferred embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidy lethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

In certain embodiments, the non-cationic lipid (e.g., one or more phospholipids and/or cholesterol) may comprise from about 9.9 mol % to about 49.9 mol % of the total lipid present in the particle. In certain embodiments, the non-cationic lipid may comprise from about 10 mol % to about 49.5 mol %, from about 13 mol % to about 49.5 mol %, from about 15 mol % to about 49.5 mol %, from about 20 mol % to about 49.5 mol %, from about 25 mol % to about 49.5 mol %, from about 30 mol % to about 49.5 mol %, from about 35 mol % to about 49.5 mol %, or from about 40 mol % to about 49.5 mol % of the total lipid present in the particle.

In some embodiments, the non-cationic lipid (e.g., one or more phospholipids and/or cholesterol) may comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

In yet other embodiments, the non-cationic lipid (e.g., one or more phospholipids and/or cholesterol) may comprise from about 10 mol % to about 45 mol %, from about 13 mol % to about 45 mol %, from about 15 mol % to about 45 mol %, from about 20 mol % to about 45 mol %, from about 25 mol % to about 45 mol %, from about 30 mol % to about 45 mol %, or from about 35 mol % to about 45 mol % of the total lipid present in the particle.

In still yet other embodiments, the non-cationic lipid (e.g., one or more phospholipids and/or cholesterol) may comprise from about 10 mol % to about 40 mol %, from about 13 mol % to about 40 mol %, from about 15 mol % to about 40 mol %, from about 20 mol % to about 40 mol %, from about 25 mol % to about 40 mol %, or from about 30 mol % to about 40 mol % of the total lipid present in the particle.

In further embodiments, the non-cationic lipid (e.g., one or more phospholipids and/or cholesterol) may comprise from about 10 mol % to about 35 mol %, from about 13 mol % to about 35 mol %, from about 15 mol % to about 35 mol %, from about 20 mol % to about 35 mol %, or from about 25 mol % to about 35 mol % of the total lipid present in the particle.

In yet further embodiments, the non-cationic lipid (e.g., one or more phospholipids and/or cholesterol) may comprise from about 10 mol % to about 30 mol %, from about 13 mol % to about 30 mol %, from about 15 mol % to about 30 mol %, from about 20 mol % to about 30 mol %, from about 10 mol % to about 25 mol %, from about 13 mol % to about 25 mol %, or from about 15 mol % to about 25 mol % of the total lipid present in the particle.

In additional embodiments, the non-cationic lipid (e.g., one or more phospholipids and/or cholesterol) may comprise (at least) about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol % (or any fraction thereof or range therein) of the total lipid present in the particle.

In certain preferred embodiments, the non-cationic lipid comprises cholesterol or a derivative thereof of from about 31.5 mol % to about 42.5 mol % of the total lipid present in the 35 particle. As a non-limiting example, a phospholipid-free lipid particle of the disclosure may comprise cholesterol or a derivative thereof at about 3 7 mol % of the total lipid present in the particle. In other preferred embodiments, a phospholipid-free lipid particle of the disclosure may comprise cholesterol or a 40 derivative thereof of from about 30 mol % to about 45 mol %, from about 30 mol % to about 40 mol %, from about 30 mol % to about 35 mol %, from about 35 mol % to about 45 mol %, from about 40 mol % to about 45 mol %, from about 32 mol % to about 45 mol %, from about 32 mol % to about 45 mol %, from about 32 mol % to about 40 mol %, from about 34 mol % to about 45 mol %, from about 34 mol % to about 42 mol %, from about 34 mol % to about 40 mol %, or about 30, 31, 32, 33, 34, 35, 36, 37, 38, 39, 40, 41, 42, 43, 44, or 45 mol % (or any fraction thereof or range therein) of the total lipid present in the particle.

In certain other preferred embodiments, the non-cationic lipid comprises a mixture of: (i) a phospholipid of from about 4 mol % to about 10 mol % of the total lipid present in the particle; and (ii) cholesterol or a derivative thereof of from about 30 mol % to about 40 mol % of the total lipid present in the particle. As a non-limiting example, a lipid particle comprising a mixture of a phospholipid and cholesterol may comprise DPPC at about 7 mol % and cholesterol at about 34 mol % of the total lipid present in the particle. In other embodiments, the non-cationic lipid comprises a mixture of: (i) a phospholipid of from about 3 mol % to about 15 mol %, from about 4 mol % to about 15 mol %, from about 4 mol % to about 12 mol %, from about 4 mol % to about 10 mol %, from about 4 mol % to about 8 mol %, from about 5 mol % to about 12 mol %, from about 5 mol % to about 9 mol %, from about 6 mol % to about 12 mol %, from about 6 mol % to about 10 mol %, or about 3, 4, 5, 6, 7, 8, 9, 10, 11, 12, 13, 14, or 15 mol % (or any fraction thereof or range therein) of the total lipid present in the particle; and (ii) cholesterol or a derivative thereof of from about 25 mol % to about 45 mol %, from about 30 mol % to about 45 mol %, from about 25 mol % to about 40 mol %, from about 30 mol % to about 40 mol %, from 5 about 25 mol % to about 35 mol %, from about 30 mol % to about 35 mol %, from about 35 mol % to about 45 mol %, from about 40 mol % to about 45 mol %, from about 28 mol % to about 40 mol %, from about 28 mol % to about 38 mol %, from about 30 mol % to about 38 mol %, from about 32 10 mol % to about 36 mol %, or about 25, 26, 27, 28, 29, 30, 31, 32, 33, 34, 35, 36, 37, 38, 39, 40, 41, 42, 43, 44, or 45 mol % (or any fraction thereof or range therein) of the total lipid present in the particle.

In further preferred embodiments, the non-cationic lipid comprises a mixture of: (i) a phospholipid of from about 10 mol % to about 30 mol % of the total lipid present in the particle; and (ii) cholesterol or a derivative thereof of from about 10 mol % to about 30 mol % of the total lipid present in the particle. As a non-limiting example, a lipid particle comprising a mixture of a phospholipid and cholesterol may comprise DPPC at about 20 mol % and cholesterol at about 20 mol % of the total lipid present in the particle.

In other embodiments, the non-cationic lipid comprises a mixture of: (i) a phospholipid of from about 10 mol % to about 30 mol %, from 25 about 10 mol % to about 25 mol %, from about 10 mol % to about 20 mol %, from about 15 mol % to about 30 mol %, from about 20 mol % to about 30 mol %, from about 15 mol % to about 25 mol %, from about 12 mol % to about 28 mol %, from about 14 mol % to about 26 mol %, or about 10, 11, 12, 13, 14, 15, 16, 17, 18, 19, 20, 21, 22, 23, 24, 25, 26, 27, 28, 29, or 30 mol % (or any fraction thereof or range therein) of the total lipid present in the particle; and (ii) cholesterol or a derivative thereof of from about 10 mol % to about 30 mol %, from about 10 mol % to about 25 mol %, from about 10 mol 35% to about 20 mol %, from about 15 mol % to about 30 mol %, from about 20 mol % to about 30 mol %, from about 15 mol % to about 25 mol %, from about 12 mol % to about 28 mol %, from about 14 mol % to about 26 mol %, or about 10, 11, 12, 13, 14, 15, 16, 17, 18, 19, 20, 21, 22, 23, 24, 25, 26, 27, 28, 29, or 30 mol % (or any fraction thereof or range therein) of the total lipid present in the particle.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In one preferred embodiment, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S-NHS), monomethoxypolyethylene glycolamine (MePEG-NH2), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH2COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly (ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In preferred embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., SNALP or SPLP) of the present disclosure can further comprise cationic poly (ethylene glycol) (PEG) lipids or CPLs (scc, e.g., Chen et al., Bioconj. Chem., 11:433-437 (2000)). Suitable SPLPs and SPLP-CPLs for use in the present disclosure, and methods of making and using SPLPs and SPLPCPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1 4, 1.5, 1.6, 1.7, 1.8, 1 9, or 2 mol % (or any fraction thereof or range therein) of the total lipid present in the particle.

In the nucleic acid-lipid particles of the present disclosure, the active agent or therapeutic agent may be fully encapsulated within the lipid portion of the particle, thereby protecting the active agent or therapeutic agent from enzymatic degradation. In preferred embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as siRNA) is complexed with the lipid portion of the particle. One of the benefits of the formulations of the present disclosure is that the lipid particle compositions are substantially non-toxic to mammals such as humans.

### Lipid: Active Agent Ratios

Typically, the nucleic acid-lipid particles of the present disclosure have a lipid: active agent (e.g., lipid: nucleic acid) ratio (mass/mass ratio) of from about 1 to about 100. In some instances, the lipid: active agent (e.g., lipid: nucleic acid) ratio (mass/mass ratio) ranges from about 1 to about 50, from about 2 to 40 about 25, from about 3 to about 20, from about 4 to about 15, or from about 5 to about 10. In certain embodiments, the lipid particles of the disclosure have a lipid: active agent (e.g., lipid: nucleic acid) ratio (mass/mass ratio) of from about 5 to about 15, e.g., about 5, 6, 7, 8, 9, 10, 11, 12, 13, 14, or 15 (or any fraction thereof or range therein).

Typically, the nucleic acid-lipid particles of the present disclosure have a mean diameter of from about 40 nm to about 150 nm. In certain embodiments, the lipid particles (e.g., SNALP) of the disclosure have a mean diameter of from about 40 nm to 50 about 130 nm, from about 40 nm to about 120 nm, from about 40 nm to about 100 nm, from about 50 nm to about 120 nm, from about 50 nm to about 100 nm, from about 60 nm to about 120 nm, from about 60 nm to about 110 nm, from about 60 nm to about 100 nm, from about 60 nm to about 90 nm, from 55 about 60 nm to about 80 nm, from about 70 nm to about 120 nm, from about 70 nm to about 110 nm, from about 70 nm to about 100 nm, from about 70 nm to about 90 nm, from about 70 nm to about 80 nm, or less than about 120 nm, 110 nm, 100 nm, 90 nm, or 80 nm (or any fraction thereof or range therein).

In certain embodiments, the nucleic acid-lipid particle comprises: (a) one or more unmodified and/or modified messenger RNA (e.g., mRNA) that encodes a functional protein (i.e., gene product); (b) a cationic lipid comprising from about 56.5 mol % to about 66.5 mol % of the total lipid present in the 65 particle; (c) a non-cationic lipid comprising from about 31.5 mol % to about 42.5 mol % of the total lipid present in the particle; and (d) a conjugated lipid that inhibits aggregation of particles comprising from about 1 mol % to about 2 mol % of the total lipid present in the particle. In certain embodiments, this nucleic acid-lipid particle is referred to herein as the “1:62” formulation. In certain embodiments, the cationic lipid is DLinDMA or DLin-K-C2-DMA (“XTC2”), the non-cationic lipid is cholesterol, and the conjugated lipid is a PEG-DAA conjugate. Although these are preferred embodiments of the 1:62 formulation, those of skill in the art will appreciate that other cationic lipids, non-cationic lipids (including other cholesterol derivatives), and conjugated lipids can be used in the 1:62 formulation as described herein.

In another embodiment of the disclosure, the nucleic acid-lipid particle comprises: (a) one or more unmodified and/or modified messenger RNA (e.g., mRNA) that encodes a functional protein (i.e., gene product); (b) a cationic lipid comprising from about 52 mol % to about 62 mol % of the total lipid present in the particle; (c) a non-cationic lipid comprising from about 36 mol % to about 47 mol % of the total lipid present in the particle; and (d) a conjugated lipid that inhibits aggregation of particles comprising from about 1 mol % to about 2 mol % of the total lipid present in the particle. This specific embodiment of nucleic acid-lipid particle is generally referred to herein as the “1:57” formulation. In this embodiment, the cationic lipid is DLinDMA or DLin-K-C2-DMA (“XTC2”), the non-cationic lipid is a mixture of a phospholipid (such as DPPC) and cholesterol, wherein the phospholipid comprises from about 5 mol % to about 9 mol % of the total lipid present in the particle (e.g., about 7.1 mol %) and the cholesterol (or cholesterol derivative) comprises from about 32 mol % to about 37 mol % of the total lipid present in the particle (e.g., about 34.3 mol %), and the PEG-lipid is a PEG-DAA (e.g., PEG-cDMA). In another preferred embodiment, the cationic lipid is DLinDMA or DLin-K-C2-DMA (“XTC2”), the non-cationic lipid is a mixture of a phospholipid (such as DPPC) and cholesterol, wherein the phospholipid comprises from about 15 mol % to about 25 mol % of the total lipid present in the particle (e.g., about 20 mol %) and the cholesterol (or cholesterol derivative) comprises from about 15 mol % to about 25 mol % of the total lipid present in the particle (e.g., about 20 mol %), and the PEG-lipid is a PEGDAA (e.g., PEG-cDMA). Those of skill in the art will appreciate that other cationic lipids, non-cationic lipids (including other phospholipids and other cholesterol derivatives), and conjugated lipids can be used in the 1:57 formulation as described herein.

In certain embodiments, the 1:62 nucleic acid-lipid particle formulation is a three-component system which is phospholipid-free and comprises about 1.5 mol % PEG-CDMA (or PEG-IDSA), about 61.5 mol % DLinDMA (or XTC2), and about 36.9 mol % cholesterol (or derivative thereof). In other embodiments, the 1:57 nucleic acid-lipid particle formulation is a four-component system which comprises about 1.4 mol % PEG-CDMA (or PEG-cDSA), about 57.1 mol % DLinDMA (or XTC2), about 7.1 mol % DPPC, and about 34.3 mol % cholesterol (or derivative thereof). In yet other preferred embodiments, the 1:57 nucleic acid-lipid particle formulation is a four-component system which comprises about 1.4 mol % PEG-CDMA (or PEG-cDSA), about 57.1 mol % DLinDMA (or XTC2), about 20 mol % DPPC, and about 20 mol % cholesterol (or derivative thereof). It should be understood that these nucleic acid-lipid particle formulations are target formulations, and that the amount of lipid (both cationic and non-cationic) present and the amount of lipid conjugate present in the nucleic acid-lipid particle formulations may vary.

### Polymer-Based Vehicles

In one aspect, the present disclosure provides a composition comprising one or more polymer-based vehicles, wherein polymer-based vehicle comprises a nucleic acid which is at least partially encapsulated within the polymer-based vehicle, wherein the nucleic acid either:
(a) encodes a protein which has a reduced abundance in a GTPase IMAP family member 5 (GIMAP5) deficient subject as compared to a healthy subject; or
(b) at least partially inhibits expression of a protein which has an increased abundance in a GIMAP5 deficient subject as compared to a healthy subject.

In certain embodiments, the polymer-based vehicle comprises polyethyleneimine (PEI). In certain embodiments, the polymer-based vehicle comprises poly-β-aminoester (PBAE). In certain embodiments, the polymer-based vehicle comprises poly-L-lysine (PLL). In certain embodiments, the polymer-based vehicle comprises chitosan. In certain embodiments, the polymer-based vehicle comprises pullulan. In certain embodiments, the polymer-based vehicle comprises dextran. In certain embodiments, the polymer-based vehicle comprises hyaluronic acid.

In certain embodiments, the polymer-based vehicle is biodegradable.

In certain embodiments, the protein which has a reduced abundance in a GTPase IMAP family member 5 (GIMAP5) deficient subject as compared to a healthy subject is an enzyme. In certain embodiments, the enzyme is GIMAP5. In certain embodiments, the nucleic acid comprises a messenger RNA (mRNA) which encodes a protein that shares at least 85% sequence homology with SEQ ID NO:1. In certain embodiments, the nucleic acid comprises a messenger RNA (mRNA) which encodes a protein that shares at least 90% sequence homology with SEQ ID NO: 1. In certain embodiments, the nucleic acid comprises a messenger RNA (mRNA) which encodes a protein that shares at least 95% sequence homology with SEQ ID NO:1. In certain embodiments, the nucleic acid comprises a messenger RNA (mRNA) which encodes a protein that shares at least 96% sequence homology with SEQ ID NO:1. In certain embodiments, the nucleic acid comprises a messenger RNA (mRNA) which encodes a protein that shares at least 97% sequence homology with SEQ ID NO:1. In certain embodiments, the nucleic acid comprises a messenger RNA (mRNA) which encodes a protein that shares at least 98% sequence homology with SEQ ID NO:1. In certain embodiments, the nucleic acid comprises a messenger RNA (mRNA) which encodes a protein that shares at least 99% sequence homology with SEQ ID NO: 1 In certain embodiments, the mRNA encodes SEQ ID NO:1.

In certain embodiments, the protein which has a reduced abundance in a GTPase IMAP family member 5 (GIMAP5) deficient subject as compared to a healthy subject is GATA4. In certain embodiments, the nucleic acid comprises a messenger RNA (mRNA) which encodes a protein that shares at least 85% sequence homology with SEQ ID NO:2. In certain embodiments, the nucleic acid comprises a messenger RNA (mRNA) which encodes a protein that shares at least 90% sequence homology with SEQ ID NO:2. In certain embodiments, the nucleic acid comprises a messenger RNA (mRNA) which encodes a protein that shares at least 95% sequence homology with SEQ ID NO:2. In certain embodiments, the nucleic acid comprises a messenger RNA (mRNA) which encodes a protein that shares at least 96% sequence homology with SEQ ID NO:2. In certain embodiments, the nucleic acid comprises a messenger RNA (mRNA) which encodes a protein that shares at least 97% sequence homology with SEQ ID NO: 2. In certain embodiments, the nucleic acid comprises a messenger RNA (mRNA) which encodes a protein that shares at least 98% sequence homology with SEQ ID NO:2. In certain embodiments, the nucleic acid comprises a messenger RNA (mRNA) which encodes a protein that shares at least 99% sequence homology with SEQ ID NO:2. In certain embodiments, the mRNA encodes SEQ ID NO:2.

In certain embodiments, the protein which has a reduced abundance in a GTPase IMAP family member 5 (GIMAP5) deficient subject as compared to a healthy subject is MAF. In certain embodiments, the nucleic acid comprises a messenger RNA (mRNA) which encodes a protein that shares at least 85% sequence homology with SEQ ID NO:3. In certain embodiments, the nucleic acid comprises a messenger RNA (mRNA) which encodes a protein that shares at least 90% sequence homology with SEQ ID NO:3. In certain embodiments, the nucleic acid comprises a messenger RNA (mRNA) which encodes a protein that shares at least 95% sequence homology with SEQ ID NO:3. In certain embodiments, the nucleic acid comprises a messenger RNA (mRNA) which encodes a protein that shares at least 96% sequence homology with SEQ ID NO:3. In certain embodiments, the nucleic acid comprises a messenger RNA (mRNA) which encodes a protein that shares at least 97% sequence homology with SEQ ID NO: 3. In certain embodiments, the nucleic acid comprises a messenger RNA (mRNA) which encodes a protein that shares at least 98% sequence homology with SEQ ID NO:3. In certain embodiments, the nucleic acid comprises a messenger RNA (mRNA) which encodes a protein that shares at least 99% sequence homology with SEQ ID NO:3. In certain embodiments, the mRNA encodes SEQ ID NO:3.

In certain embodiments, the protein which has a reduced abundance in a GTPase IMAP family member 5 (GIMAP5) deficient subject as compared to a healthy subject is MEIS2. In certain embodiments, the nucleic acid comprises a messenger RNA (mRNA) which encodes a protein that shares at least 85% sequence homology with SEQ ID NO:4. In certain embodiments, the nucleic acid comprises a messenger RNA (mRNA) which encodes a protein that shares at least 90% sequence homology with SEQ ID NO:4. In certain embodiments, the nucleic acid comprises a messenger RNA (mRNA) which encodes a protein that shares at least 95% sequence homology with SEQ ID NO:4. In certain embodiments, the nucleic acid comprises a messenger RNA (mRNA) which encodes a protein that shares at least 96% sequence homology with SEQ ID NO:4. In certain embodiments, the nucleic acid comprises a messenger RNA (mRNA) which encodes a protein that shares at least 97% sequence homology with SEQ ID NO: 4. In certain embodiments, the nucleic acid comprises a messenger RNA (mRNA) which encodes a protein that shares at least 98% sequence homology with SEQ ID NO:4. In certain embodiments, the nucleic acid comprises a messenger RNA (mRNA) which encodes a protein that shares at least 99% sequence homology with SEQ ID NO:4. In certain embodiments, the mRNA encodes SEQ ID NO:4.

In certain embodiments, the nucleic acid at least partially inhibits expression of a protein which has an increased abundance in a GIMAP5 deficient subject as compared to a healthy subject.

In certain embodiments, the protein which has an increased abundance in a GIMAP5 deficient subject is PDGFβ. In certain embodiments, the protein which has an increased abundance in a GIMAP5 deficient subject is VEGFα. In certain embodiments, the protein which has an increased abundance in a GIMAP5 deficient subject is APLN. In certain embodiments, the protein which has an increased abundance in a GIMAP5 deficient subject is MYC. In certain embodiments, the protein which has an increased abundance in a GIMAP5 deficient subject is GATA6.

In certain embodiments, the nucleic acid comprises a small interfering RNA (siRNA).

In certain embodiments, the siRNA at least partially inhibits expression of a protein that shares at least 85% sequence homology with SEQ ID NO:5. In certain embodiments, the siRNA at least partially inhibits expression of a protein that shares at least 90% sequence homology with SEQ ID NO:5. In certain embodiments, the siRNA at least partially inhibits expression of a protein that shares at least 95% sequence homology with SEQ ID NO:5. In certain embodiments, the siRNA at least partially inhibits expression of a protein that shares at least 96% sequence homology with SEQ ID NO:5. In certain embodiments, the siRNA at least partially inhibits expression of a protein that shares at least 97% sequence homology with SEQ ID NO: 5. In certain embodiments, the siRNA at least partially inhibits expression of a protein that shares at least 98% sequence homology with SEQ ID NO:5. In certain embodiments, the siRNA at least partially inhibits expression of a protein that shares at least 99% sequence homology with SEQ ID NO:5. In certain embodiments, the siRNA at least partially inhibits expression of a protein with sequence SEQ ID NO:5.

In certain embodiments, the siRNA at least partially inhibits expression of a protein that shares at least 85% sequence homology with SEQ ID NO:6. In certain embodiments, the siRNA at least partially inhibits expression of a protein that shares at least 90% sequence homology with SEQ ID NO:6. In certain embodiments, the siRNA at least partially inhibits expression of a protein that shares at least 95% sequence homology with SEQ ID NO:6. In certain embodiments, the siRNA at least partially inhibits expression of a protein that shares at least 96% sequence homology with SEQ ID NO:6. In certain embodiments, the siRNA at least partially inhibits expression of a protein that shares at least 97% sequence homology with SEQ ID NO: 6. In certain embodiments, the siRNA at least partially inhibits expression of a protein that shares at least 98% sequence homology with SEQ ID NO:6. In certain embodiments, the siRNA at least partially inhibits expression of a protein that shares at least 99% sequence homology with SEQ ID NO:6. In certain embodiments, the siRNA at least partially inhibits expression of a protein with sequence SEQ ID NO:6.

In certain embodiments, the siRNA at least partially inhibits expression of a protein that shares at least 85% sequence homology with SEQ ID NO:7. In certain embodiments, the siRNA at least partially inhibits expression of a protein that shares at least 90% sequence homology with SEQ ID NO:7. In certain embodiments, the siRNA at least partially inhibits expression of a protein that shares at least 95% sequence homology with SEQ ID NO:7. In certain embodiments, the siRNA at least partially inhibits expression of a protein that shares at least 96% sequence homology with SEQ ID NO:7. In certain embodiments, the siRNA at least partially inhibits expression of a protein that shares at least 97% sequence homology with SEQ ID NO: 7. In certain embodiments, the siRNA at least partially inhibits expression of a protein that shares at least 98% sequence homology with SEQ ID NO:7. In certain embodiments, the siRNA at least partially inhibits expression of a protein that shares at least 99% sequence homology with SEQ ID NO:7. In certain embodiments, the siRNA at least partially inhibits expression of a protein with sequence SEQ ID NO:7.

In certain embodiments, the siRNA at least partially inhibits expression of a protein that shares at least 85% sequence homology with SEQ ID NO:8. In certain embodiments, the siRNA at least partially inhibits expression of a protein that shares at least 90% sequence homology with SEQ ID NO:8. In certain embodiments, the siRNA at least partially inhibits expression of a protein that shares at least 95% sequence homology with SEQ ID NO:8. In certain embodiments, the siRNA at least partially inhibits expression of a protein that shares at least 96% sequence homology with SEQ ID NO:8. In certain embodiments, the siRNA at least partially inhibits expression of a protein that shares at least 97% sequence homology with SEQ ID NO: 8. In certain embodiments, the siRNA at least partially inhibits expression of a protein that shares at least 98% sequence homology with SEQ ID NO:8. In certain embodiments, the siRNA at least partially inhibits expression of a protein that shares at least 99% sequence homology with SEQ ID NO:8. In certain embodiments, the siRNA at least partially inhibits expression of a protein with sequence SEQ ID NO:8.

In certain embodiments, the siRNA at least partially inhibits expression of a protein that shares at least 85% sequence homology with SEQ ID NO:9. In certain embodiments, the siRNA at least partially inhibits expression of a protein that shares at least 90% sequence homology with SEQ ID NO:9. In certain embodiments, the siRNA at least partially inhibits expression of a protein that shares at least 95% sequence homology with SEQ ID NO:9. In certain embodiments, the siRNA at least partially inhibits expression of a protein that shares at least 96% sequence homology with SEQ ID NO:9. In certain embodiments, the siRNA at least partially inhibits expression of a protein that shares at least 97% sequence homology with SEQ ID NO: 9. In certain embodiments, the siRNA at least partially inhibits expression of a protein that shares at least 98% sequence homology with SEQ ID NO:9. In certain embodiments, the siRNA at least partially inhibits expression of a protein that shares at least 99% sequence homology with SEQ ID NO:9. In certain embodiments, the siRNA at least partially inhibits expression of a protein with sequence SEQ ID NO:9.

Cellular transport of nucleic acids is hampered by cell membranes, and accordingly, delivery vehicles are essential for the realization of gene therapy to combat any of a number of genetic diseases. In certain aspects, contact of nucleic acids and cationic polymers, including but not limited to polyethyleneimine are capable of condensing DNA to nanoparticles, thereby facilitating gene delivery.

Exemplary cationic species utilized as vehicles for gene delivery include, but are not limited to, organic cations, including cationic lipids, polyamine-based polymers, carbohydrate-based polymers (e.g., chitosan-based polymers), dendrimers, and polyethyleneimine (PEI). Interactions of the aforementioned polymeric species may induce formation of compact nucleic acid polyplexes which protect the nucleic acids from nucleases and maintain stability and integrity of the sequences (i.e., partial or full encapsulation of the nucleic acid by the polymer-based vehicle).

A detailed description of polymer-based vehicles suitable for delivery of nucleic acids is provided in T. J. Thomas et al. (Molecules 2019, 24 (3744): 1-24), which is incorporated herein by reference in its entirety.

### Nucleic Acids

In certain embodiments, lipid particles of the present disclosure are associated with a nucleic acid, resulting in a nucleic acid-lipid particle (e.g., SNALP). In certain embodiments, the nucleic acid is fully encapsulated in the lipid particle.

The nucleic acid that is present in a lipid-nucleic acid particle according to this disclosure includes any form of nucleic acid that is known. The nucleic acids used herein can be single-stranded DNA or RNA, or double-stranded DNA or RNA, or DNA-RNA hybrids. Examples of double-stranded DNA are described herein and include, e.g., structural genes, genes including control and termination regions, and self-replicating systems such as viral or plasmid DNA. Single-stranded nucleic acids include, e.g., mRNA, antisense oligonucleotides, ribozymes, mature miRNA, and triplex-forming oligonucleotides.

Nucleic acids of the disclosure may be of various lengths, generally dependent upon the particular form of nucleic acid. For example, in particular embodiments, plasmids or genes may be from about 1,000 to about 100,000 nucleotide residues in length. In particular embodiments, oligonucleotides may range from about 10 to about 100 nucleotides in length. In various related embodiments, oligonucleotides, both single-stranded, double-stranded, and triple-stranded, may range in length from about 10 to about 60 nucleotides, from about 15 to about 60 nucleotides, from about 20 to about 50 nucleotides, from about 15 to about 30 nucleotides, or from about 20 to about 30 nucleotides in length.

In certain embodiments, the nucleic acid is mRNA. In certain embodiments of the disclosure, mRNA can comprise one or more modifications that confer stability to the mRNA (e.g., compared to a wild-type or native version of the mRNA) and may also comprise one or more modifications relative to the wild-type which correct a defect implicated in the associated aberrant expression of the protein. For example, the nucleic acids of the present disclosure may comprise modifications to one or both of the 5′ and 3′ untranslated regions. Such modifications may include, but are not limited to, the inclusion of a partial sequence of a cytomegalovirus (CMV) immediate-early 1 (IE1) gene, a poly A tail, a Cap1 structure, or a sequence encoding human growth hormone (hGH)). In some embodiments, the mRNA is modified to decrease mRNA immunogenecity.

The mRNA in the compositions of the disclosure may encode, for example, a hormone, enzyme, receptor, polypeptide, peptide or other protein of interest that is normally expressed in a subject. In one embodiment of the disclosure, the mRNA may optionally have chemical or biological modifications which, for example, improve the stability and/or half-life of such mRNA or which improve or otherwise facilitate protein production.

One or more unique mRNA can be co-delivered to target cells, for example, by combining two unique mRNAs into a single transfer vehicle. In one embodiment of the present disclosure, a therapeutic first mRNA, and a therapeutic second mRNA, may be formulated in a single transfer vehicle and administered. The present disclosure also contemplates co-delivery and/or co-administration of a therapeutic first mRNA and a second nucleic acid to facilitate and/or enhance the function or delivery of the therapeutic first mRNA. For example, such a second nucleic acid (e.g., exogenous or synthetic mRNA) may encode a membrane transporter protein that upon expression (e.g., translation of the exogenous or synthetic mRNA) facilitates the delivery or enhances the biological activity of the first mRNA. Alternatively, the therapeutic first mRNA may be administered with a second nucleic acid that functions as a “chaperone” for example, to direct the folding of either the therapeutic first mRNA.

Also contemplated are methods that provide for the delivery of one or more therapeutic nucleic acids to treat a single disorder or deficiency, wherein each such therapeutic nucleic acid functions by a different mechanism of action. For example, the compositions of the present disclosure may comprise a therapeutic first mRNA which, for example, is administered to correct an endogenous protein or enzyme deficiency, and which is accompanied by a second nucleic acid (i.e., a siRNA), which is administered to deactivate or “knock-down” a malfunctioning and/or overexpressed endogenous nucleic acid and its protein or enzyme product. Such “second” nucleic acids may encode, for example mRNA or siRNA.

Upon transfection, a natural mRNA in the compositions of the disclosure may decay with a half-life of between 30 minutes and several days. The mRNA in the compositions of the disclosure preferably retain at least some ability to be translated, thereby producing a functional protein or enzyme. Accordingly, the disclosure provides compositions comprising a stabilized mRNA. In some embodiments of the disclosure, the activity of the mRNA is prolonged over an extended period of time. For example, the activity of the mRNA may be prolonged such that the compositions of the present disclosure are administered to a subject on a semi-weekly or bi-weekly basis, or more preferably on a monthly, bi-monthly, quarterly or an annual basis. The extended or prolonged activity of the mRNA of the present disclosure, is directly related to the quantity of functional protein or enzyme produced from such mRNA. Similarly, the activity of the compositions of the present disclosure may be further extended or prolonged by modifications made to improve or enhance translation of the mRNA. Furthermore, the quantity of functional protein or enzyme produced by the target cell is a function of the quantity of mRNA delivered to the target cells and the stability of such mRNA. To the extent that the stability of the mRNA of the present disclosure may be improved or enhanced, the half-life, the activity of the produced secreted protein or enzyme and the dosing frequency of the composition may be further extended.

Accordingly, in some embodiments, the mRNA and/or siRNA in the compositions of the disclosure comprise at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present disclosure, and particularly with respect to the mRNA, refer to increased or enhanced resistance to degradation by, for example nucleases (e.g., endonucleases or exonucleases) which are normally capable of degrading such mRNA. Increased stability can include, for example, less sensitivity to hydrolysis or other destruction by endogenous enzymes (e.g., endonucleases or exonucleases) or conditions within the target cell or tissue, thereby increasing or enhancing the residence of such mRNA in the target cell, tissue, subject and/or cytoplasm. The stabilized mRNA molecules provided herein demonstrate longer half-lives relative to their naturally occurring, unmodified counterparts (e.g., the wild-type version of the mRNA). Also contemplated by the terms “modification” and “modified” as such terms related to the mRNA of the present disclosure are alterations which improve or enhance translation of mRNA nucleic acids, including for example, the inclusion of sequences which function in the initiation of protein translation (e.g., the Kozac consensus sequence).

### Adeno-Associated Viral (AAV) Vectors

In one aspect, the present disclosure provides a recombinant viral vector, the vector comprising:
(a) an expression cassette comprising a nucleic acid sequence encoding a protein which has a reduced abundance in a GTPase IMAP family member 5 (GIMAP5) deficient subject as compared to a healthy subject; and
(b) an expression control sequence operably linked to the nucleic acid.

In certain embodiments, the protein which has a reduced abundance in a GTPase IMAP family member 5 (GIMAP5) deficient subject as compared to a healthy subject is an enzyme. In certain embodiments, the enzyme is GIMAP5. In certain embodiments, the nucleic acid comprises a DNA sequence which encodes a protein that shares at least 85% sequence homology with SEQ ID NO:1. In certain embodiments, the nucleic acid comprises a DNA sequence which encodes a protein that shares at least 90% sequence homology with SEQ ID NO:1. In certain embodiments, the nucleic acid comprises a DNA sequence which encodes a protein that shares at least 95% sequence homology with SEQ ID NO: 1. In certain embodiments, the nucleic acid comprises a DNA sequence which encodes a protein that shares at least 96% sequence homology with SEQ ID NO:1. In certain embodiments, the nucleic acid comprises a DNA sequence which encodes a protein that shares at least 97% sequence homology with SEQ ID NO:1. In certain embodiments, the nucleic acid comprises a DNA sequence which encodes a protein that shares at least 98% sequence homology with SEQ ID NO:1. In certain embodiments, the nucleic acid comprises a DNA sequence which encodes a protein that shares at least 99% sequence homology with SEQ ID NO:1 In certain embodiments, the DNA sequence encodes SEQ ID NO:1.

In certain embodiments, the protein which has a reduced abundance in a GTPase IMAP family member 5 (GIMAP5) deficient subject as compared to a healthy subject is GATA4. In certain embodiments, the nucleic acid comprises a DNA sequence which encodes a protein that shares at least 85% sequence homology with SEQ ID NO:2. In certain embodiments, the nucleic acid comprises a DNA sequence which encodes a protein that shares at least 90% sequence homology with SEQ ID NO:2. In certain embodiments, the nucleic acid comprises a DNA sequence which encodes a protein that shares at least 95% sequence homology with SEQ ID NO: 2. In certain embodiments, the nucleic acid comprises a DNA sequence which encodes a protein that shares at least 96% sequence homology with SEQ ID NO:2. In certain embodiments, the nucleic acid comprises a DNA sequence which encodes a protein that shares at least 97% sequence homology with SEQ ID NO:2. In certain embodiments, the nucleic acid comprises a DNA sequence which encodes a protein that shares at least 98% sequence homology with SEQ ID NO:2. In certain embodiments, the nucleic acid comprises a DNA sequence which encodes a protein that shares at least 99% sequence homology with SEQ ID NO:2 In certain embodiments, the DNA sequence encodes SEQ ID NO:2.

In certain embodiments, the protein which has a reduced abundance in a GTPase IMAP family member 5 (GIMAP5) deficient subject as compared to a healthy subject is MAF. In certain embodiments, the nucleic acid comprises a messenger RNA (mRNA) which encodes a protein that shares at least 85% sequence homology with SEQ ID NO:3. In certain embodiments, the nucleic acid comprises a messenger RNA (mRNA) which encodes a protein that shares at least 90% sequence homology with SEQ ID NO:3. In certain embodiments, the nucleic acid comprises a messenger RNA (mRNA) which encodes a protein that shares at least 95% sequence homology with SEQ ID NO:3. In certain embodiments, the nucleic acid comprises a messenger RNA (mRNA) which encodes a protein that shares at least 96% sequence homology with SEQ ID NO:3. In certain embodiments, the nucleic acid comprises a messenger RNA (mRNA) which encodes a protein that shares at least 97% sequence homology with SEQ ID NO: 3. In certain embodiments, the nucleic acid comprises a messenger RNA (mRNA) which encodes a protein that shares at least 98% sequence homology with SEQ ID NO:3. In certain embodiments, the nucleic acid comprises a messenger RNA (mRNA) which encodes a protein that shares at least 99% sequence homology with SEQ ID NO:3. In certain embodiments, the mRNA encodes SEQ ID NO:3.

In certain embodiments, the protein which has a reduced abundance in a GTPase IMAP family member 5 (GIMAP5) deficient subject as compared to a healthy subject is MEIS2. In certain embodiments, the nucleic acid comprises a messenger RNA (mRNA) which encodes a protein that shares at least 85% sequence homology with SEQ ID NO:4. In certain embodiments, the nucleic acid comprises a messenger RNA (mRNA) which encodes a protein that shares at least 90% sequence homology with SEQ ID NO:4. In certain embodiments, the nucleic acid comprises a messenger RNA (mRNA) which encodes a protein that shares at least 95% sequence homology with SEQ ID NO:4. In certain embodiments, the nucleic acid comprises a messenger RNA (mRNA) which encodes a protein that shares at least 96% sequence homology with SEQ ID NO:4. In certain embodiments, the nucleic acid comprises a messenger RNA (mRNA) which encodes a protein that shares at least 97% sequence homology with SEQ ID NO: 4. In certain embodiments, the nucleic acid comprises a messenger RNA (mRNA) which encodes a protein that shares at least 98% sequence homology with SEQ ID NO:4. In certain embodiments, the nucleic acid comprises a messenger RNA (mRNA) which encodes a protein that shares at least 99% sequence homology with SEQ ID NO:4. In certain embodiments, the mRNA encodes SEQ ID NO:4.

In certain embodiments, the recombinant viral vector is an Adeno-associated virus (AAV) vector.

In one embodiment, an AAV2-derived ITR sequence, or a deleted form thereof (ΔITR), is used for convenience and for accelerated regulatory approval. However, ITRs from other AAV sources may be selected. If the source of the ITR is from AAV2 and the AAV capsid is from another source of AAV, the resulting vector can be referred to as a pseudoform.

Expression cassettes for AAV vectors typically include AAV 5′-ITRs, coding sequences and arbitrary control sequences, as well as AAV 3′-ITRs. However, other arrangements of these elements may be appropriate. A shortened version of the 5′-ITR called ΔITR, which lacks the D sequence and the terminal resolution site (trs), has been described. In other embodiments, the full length AAV 5′-ITR and 3′-ITR are used.

The expression cassette usually contains, for example, a promoter sequence as part of an expression control sequence located between the selected 5′-ITR sequence and the coding sequence.

In addition to promoters, expression cassettes and/or vectors include one or more other suitable transcription initiation, termination, enhancer sequences, efficient RNA processing signals such as splicing and polyadenylation (polyA) signals. It may contain a sequence that stabilizes mRNA; a sequence that enhances translation efficiency (i.e., a Kozak consensus sequence); a sequence that enhances protein stability; and, if desired, a sequence that enhances the secretion of the encoding product.

Examples of suitable poly A sequences include, for example, SV40, SV50, bovine growth hormone (bGH), human growth hormone, and synthetic poly A.

Examples of suitable enhancers include, for example, α-fetoprotein enhancer, TTR minimum promoter/enhancer, LSP (TH-binding globulin promoter/α-microglobulin/bikunin enhancer). In one embodiment, the expression cassette comprises one or more expression enhancers. In one embodiment, the expression cassette contains two or more expression enhancers. These enhancers may be the same or different from each other. The enhancer can be present in two copies located adjacent to each other. Alternatively, the dual copy of the enhancer can be separated by one or more sequences. In yet another embodiment, the expression cassette further contains an intron, such as a Promega intron. Other suitable introns include those known in the art, such as those described in International Patent Application No. WO 2011/126808, which is incorporated herein by reference in its entirety for all purposes.

Recombinant AAV viral vectors are well suited for delivery of the coding sequences described herein. Such AAV vectors are ITRs derived from the same AAV source as the capsid. Alternatively, the AAV ITR may be derived from an AAV source different from that supplying the capsid.

Yet other promoters, including tissue-specific promoters, may be selected. Methods for making and isolating AAV viral vectors suitable for delivery to subjects are known in the art. For example, U.S. Patent Application Publication No. US2007/0036760 (Feb. 15, 2007), U.S. Pat. Nos. 7,790,449; 7,282,199; 7,588,772; and International Publication Nos. WO2003/042397; WO2005/033321; WO2006/11689; all of which are incorporated herein by reference in their entireties for all purposes. The sequence of AAV8 and the method for producing a vector based on AAV8 capsid are described in U.S. Pat. Nos. 7,282,199; 7,790,449; and 8,318,480; all of which are incorporated herein by reference in their entireties.

### Methods

In another aspect, the present disclosure provides a method of treating, ameliorating and/or preventing liver disease and/or portal hypertension in a subject, the method comprising administering to the subject in need thereof a therapeutically effective amount of the pharmaceutical composition comprising one or more nucleic acid-lipid particles and a pharmaceutically acceptable carrier, as described herein.

In one aspect, the present disclosure provides a method of treating, ameliorating and/or preventing liver disease and/or portal hypertension in a subject, the method comprising administering to the subject in need thereof a therapeutically effective amount of the recombinant viral vector described herein.

In certain embodiments, the subject is GIMAP5 deficient. In certain embodiments, the subject has a loss-of-function (LOF) mutation in Gimap5.

In certain embodiments, formation of a basement membrane in a liver endothelial cell of a subject is prevented, reduced, and/or reversed.

In certain embodiments, formation of a basement membrane in liver sinusoidal endothelial cells (LSECs) of a subject is prevented, reduced, and/or reversed. In certain embodiments, formation of a basement membrane in liver macrovascular endothelial cells of a subject is prevented, reduced, and/or reversed. In certain embodiments, formation of a basement membrane in liver lymphatic endothelial cells of a subject is prevented, reduced, and/or reversed.

In certain embodiments, loss of one or more fenestrations in liver endothelial cells of a subject is prevented, reduced, and/or prevented.

In certain embodiments, loss of one or more fenestrations in liver sinusoidal endothelial cell (LSECs) of a subject is prevented, reduced, and/or reversed. In certain embodiments, loss of one or more fenestrations in liver macrovascular endothelial cells of a subject is prevented, reduced, and/or reversed. In certain embodiments, loss of one or more fenestrations in liver lymphatic endothelial cells of a subject is prevented, reduced, and/or reversed.

In certain embodiments, the subject is a mammal. In certain embodiments, the mammal is a human.

### Pharmaceutical Compositions

The present disclosure provides pharmaceutical compositions comprising a nucleic acid-lipid particle and a pharmaceutically acceptable carrier. The present disclosure further provides a pharmaceutical composition comprising a recombinant viral vector and a pharmaceutically acceptable carrier.

Such a pharmaceutical composition may consist of at least one composition or vector of the invention, in a form suitable for administration to a subject, or the pharmaceutical composition may comprise at least one composition or vector, and one or more pharmaceutically acceptable carriers, one or more additional ingredients, or any combinations of these. At least one composition or vector of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMINR), solubilized gelatins (e.g., GELOFUSINER), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition and/or recombinant viral vector) necessary to achieve a therapeutic effect may vary according to factors such as the activity of the particular therapeutic employed; the time of administration; the rate of excretion of the composition and/or recombinant viral vector; the duration of the treatment; other drugs, compounds or materials used in combination with the composition and/or recombinant viral vector; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition and/or recombinant viral vector of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition and/or recombinant viral vector without undue experimentation.

The composition and/or recombinant viral vector may be administered to an animal as frequently as several times daily, or it may be administered less frequently, such as once a day, once a week, once every two weeks, once a month, or even less frequently, such as once every several months or even once a year or less. It is understood that the amount of composition and/or recombinant viral vector dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition and/or recombinant viral vector calculated to produce the desired therapeutic effect in association with the required pharmaceutical vehicle. The dosage unit forms of the disclosure are dictated by and directly dependent on (a) the unique characteristics of the therapeutic composition and/or recombinant viral vector and the particular therapeutic effect to be achieved, and (b) the limitations inherent in the art of compounding/formulating such a therapeutic composition and/or recombinant viral vector for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) and/or recombinant viral vector(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition and/or recombinant viral vector of the disclosure is from about 0.5 μg and about 5,000 mg. In some embodiments, a dose of active agent present in the composition and/or recombinant viral vector of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition and/or recombinant viral vector of the disclosure, alone or in combination with a second pharmaceutical agent; and instructions for using the compound to treat, prevent, or reduce one or more symptoms of a disease or disorder in a patient

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intrahepatic, intravenous, intraperitoneal, intramuscular, intrasternal injection, loco-regional delivery, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

The present disclosure is now described with reference to the following Examples. These Examples are provided for the purpose of illustration only and the present disclosure should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, point out specific embodiments of the present disclosure, and are not to be construed as limiting in any way the remainder of the present disclosure.

### Materials and Methods

### Exome Sequencing Analysis

Genomic DNA was obtained from peripheral blood leukocytes using standard methods. Exome sequence data were aligned to the reference human genome (build 19) using the Burrows-Wheeler Aligner, and variants were called using the Genome Analysis Toolkit. Variants with minor allele frequency <1% in the gnomAD databases (125,748 exome sequences of various ethnicities) were selected and annotated using ANNOVAR.

### Sanger Sequencing of Genomic DNA

Sanger sequencing of the identified GIMAP5 variants was performed by PCR amplification of genomic DNA of all available affected individuals and their parents using the following forward and reverse primers: kindred 1, chr: 7:150439367, 59-AAGATAACTTGTCTGCAACACCA-39 (forward) (SEQ ID NO:10) and 59-GTAGCAGTC CCCGATGTTCT-39 (reverse) (SEQ ID NO:11); kindred 2, chr7: 150439894, 59-GGG GAGGACGTTCATAGCTT-39 (forward) (SEQ ID NO:12) and 59-TGCTCCAGGGTC CAGAGAT-39 (reverse) (SEQ ID NO: 13); kindred 3, chr7: 150439553, 59-GATAAC TTGTCTGCAACACCA-39 (forward) (SEQ ID NO:14) and 59-TGTCCTGAGCAG TGAAACGC-39 (reverse) (SEQ ID NO: 15); kindred 4, chr7: 150439838, 59-ACA TGGAACGGGAGGAAAGTC-39 (forward) (SEQ ID NO:16) and 59-CACGAGGATCTGAGTCTGAATTG-39 (reverse) (SEQ ID NO:17). Nomenclature of the GIMAP5 variant is based on National Center for Biotechnology Information reference sequence NM 018384.

### Orthologues and Other Human GIMAPs

Full-length orthologues of GIMAP5 protein sequences from several species and related human GIMAP family members (GIMAPs 1, 2, 4, 6, 7, and 8) were obtained from GenBank. Protein sequences were aligned using the Clustal Omega algorithm.

### Mice

All mice were maintained under specific pathogen-free conditions at Yale Animal Resources Center and used according to a protocol approved by the Yale University Institutional Animal Care and Use Committee. Gimap5sph/sph mice are available through the Mutant Mouse Resource & Research Centers (stock no. 030019-UCD). Rag1−/−mice were purchased from The Jackson Laboratory (stock no. 002216). Gimap5flx/flx mice were crossed with B6.Cg-Commd10Tg (Vav1-icre) A2Kio/J (purchased from The Jackson Laboratory; stock no. 008610) or with Cdh5 (PAC)-CreERT2 strain. This strain was obtained with the permission of Dr. Ralf Adams (Max Planck Institute for Molecular Biomedicine, Muenster, Germany). Cdh5-ERT2 cre transgene expression was induced by administration of tamoxifen (Sigma-Aldrich; T5648). Tamoxifen was dissolved at a concentration of 10 mg/ml in corn oil (Sigma-Aldrich; C8267). Mice of both sexes were injected i.p. with 75 μg tamoxifen per gram of body weight daily for 5 consecutive days starting at 4 weeks of age, followed by a 9 day pause, and treated for another round of five consecutive injections at 75 μg/g. Animals were analyzed within 1-4 weeks after the final injection.

### Liver Endothelial and Kupffer Cell Isolation

Mice were euthanized in accordance with institutional animal care and use committee guidelines. The liver was flushed with PBS (1×, filtered) via the portal vein, followed by collagenase type 2 (Worthington Biochemical Corporation; LS004177) at a concentration of 1.5 mg/ml. The whole liver was excised, briefly rinsed in 1×PBS, then placed into 10 ml of prewarmed collagenase (1.5 mg/ml, 37° C.). The liver was minced into small pieces, then digested at 37° C. for 30 min. The minced liver was dissociated into single-cell suspension by gentle pipetting, then passed over a 40-μm filter. Hepatocytes were pelleted and re-moved by a series of low-speed centrifugations (60 g). Non-parenchymal cells remaining in the supernatant were pelleted by high-speed spin (350 g) and washed in RPMI media. Non-parenchymal cells were run through a Percoll gradient to enrich for endothelial cells. A density gradient was created by layering 5 ml of 50% Percoll, followed by 6.6 ml of 25% Percoll, then 3.3 ml of cell suspension in RPMI. The 100% Percoll stock was prepared by adding one part 10× PBS to nine parts Percoll (Sigma-Aldrich; P4937) and adjusting pH to 7.4. Cells were spun through the gradient at 900 g for 20 min with no brake to separate the endothelial cell fraction. The endothelial/Kupffer cell fraction was collected from the interface between the 50% and 25% Percoll layers, then washed in RPMI media and counted.

### Murine Histology

Liver tissue was collected and immediately fixed in 10% buffered formalin solution, followed by paraffin embedding. H&E, reticulin, trichrome, and CD34 staining were performed on 5 μm sections from the paraffin-embedded tissue blocks and imaged on an Olympus BX51 light microscope.

### Flow Cytometry and Cell Sorting

Endothelial cells were isolated as described above, and flow cytometry was performed on live cells. All antibody dilution and wash steps were performed using FACS buffer (2% FBS, 1 mM EDTA, 0.1% NaN3). Staining was performed on 96-well round-bottomed plates with ˜1 million cells/well using purified rat anti-mouse CD16/CD32 Mouse BD Fc Block (BD Pharmingen; 553141), followed by incubation in fluorochrome-conjugated primary antibodies for 20 min on ice. All the following anti-bodies were purchased from BioLegend and used at 1:100 dilution: allophycocyanin/cyanine 7 anti-mouse CD45 (103116), BV605 anti-mouse CD31 (102427), PE/Cy5 anti-mouse CD34 (119311), FITC anti-mouse Ly-6A (Sca-1; 108105), allophycocyanin anti-mouse F4/80 (123116), and Alexa Fluor 488 anti-mouse CD115 (CSF-IR; 135512). DAPI dye was used to determine viability. Liver endothelial cells were DAPI-CD45-CD31+, and these cells were stained for Sca-1 and Cd34. Of note, Sca-1 has been shown to be expressed in LSECs and in bone marrow arterial and sinusoidal endothelial cells. Flow cytometry experiments were performed on the LSR II flow cytometer. Cell sorting was performed on a BD FACSAria II platform. Analysis was performed using FlowJo software.

### Real-Time PCR TaqMan Assay

RNA was isolated from endothelial cells, Kupffer cells, splenocytes, and hepatocytes using RNeasy Micro Plus or RNeasy Mini Plus kits. cDNA synthesis was performed using the SuperScript III cDNA Synthesis Kit (Invitrogen) and oligo (dT) 12-18 primers (Invitrogen) following a standard protocol. Real-time PCR was performed using FastStart Universal Probe Master Mix (Roche; 4913957001) and TaqMan assays for mouse Gimap5 (Thermo Fisher Scientific; Mm00658393_m1) and GAPDH (Thermo Fisher Scientific; Mm99999915_g1) on the ABI 7500 Real Time PCR System.

### Immunoblotting

Protein lysates were prepared from cell pellets following direct isolation or cell sorting using Pierce radioimmunoprecipitation assay lysis buffer (Thermo Fisher Scientific; 89900) and standard methods. Lysates were run on 10% Mini PROTEAN TGX precast gels (Bio-Rad Laboratories) and transferred onto 0.2-μm nitrocellulose membranes (Bio-Rad Laboratories; 162-0146). Membranes were blocked in 5% milk and immunoblotted with primary antibody to GIMAP5 (MAC421) overnight at 4° C. or with GAPDH (In-vitrogen; AM4300) for 1 h at room temperature. Secondary antibodies used were ECL anti-rat IgG HRP-linked whole antibody (GE Healthcare; NA935) and ECL peroxidase-labeled anti-mouse antibody (GE Healthcare; NA931), respectively. Immunostaining was detected using Amersham ECL and ECL Prime Western Blotting detection reagents (GE Healthcare).

### Confocal Microscopy

Endothelial cells were isolated and sorted as described above. Cells were then spun onto microscope slides coated in 30% FBS at 800 revolutions per minute for 3 min using a cytocentrifuge (Thermo Scientific Cytospin 4). Cells were immediately fixed in 4% paraformaldehyde for 15 min. Background fluorescence was quenched with 100 mM ammonium chloride for 10 min and then permeabilized with 0.1% Triton X-100 for 15 min. PBS with 10% FBS was then used as a blocking solution for 1 h. Cells were then incubated overnight using anti-Gimap5 (MAC421) and anti-Lamp-1 (Abcam; ab208943). Secondary antibodies donkey anti-rat A488 (Jackson ImmunoResearch; 712-545-150) and donkey anti-rabbit A594 (Jackson ImmunoResearch; 711-585-152) were incubated for 1 h at room temperature. Cells were then stained for cytochrome-c using a directly conjugated Alexa Fluor 647 anti-body (BD Biosciences; 558709) for 2 h at room temperature. DAPI was used to counterstain the nucleus, and slides were imaged on a Leica TCS SP8 confocal microscope.

scRNA-Seq and Analysis

Liver endothelial (DAPI-CD45-CD31+) cells isolated from one Gimap5sph/sph adult mouse and one Gimap5sph/+ adult mouse were sorted as outlined above and processed as follows: scRNA-seq library was generated using Chromium Single Cell 39 Reagent Kits version 3 (10× Genomics) following the manufacturer's protocol. Libraries were sequenced on an Illumina NovaSeq 6000 instrument using an S4 flow cell. Cell Ranger version 1.3 software (10× Genomics) was used to process Chromium Single Cell 39 RNA-seq output data into a gene-cell data matrix. Seurat version 3.1.4 (Butler et al., 2018) was used for quality control, and cells with <200 expressed genes or >10% mitochondrial gene percentages were excluded to enrich high-quality cells. Cells were also discarded if Pecam 1 was not expressed to elevate the fraction of endothelial cells. The data were log transformed and normalized by the total expression. Principal component (PC) analysis was then performed using the top 2,000 variable genes to determine significant PCs. 20 statistically significant PCs were selected based on the JackStraw plot and provided as input for constructing a shared-nearest-neighbors graph based on the Euclidean distance. Cells were clustered by the Louvain method with a resolution parameter 0.4. Uniform Manifold Approximation and Projection was used to visualize clustering results. Marker genes that define each cluster were identified by comparing cells in a specific cluster with ones in all other clusters using the Seurat package likelihood ratio test. Statistically significant differentially expressed genes between samples were identified in a pairwise manner using DESeq2, which is based on the negative binomial distribution model. Four subsets of liver endothelial cells were defined based on gene expression profiles as follows (Kalucka et al., 2020): (1) LSECs expressing Clec4g and Dnase113; (2) lymphatic endothelial cells expressing Ccl21a, Prss23, Ifi2712a, andTimp2a; (3) macrovascular venous endothelial cells expressing Rspo3; and (4) macrovascular arterial endothelial cells expressing high levels of Sdc1, Clu, andEhd4.CECsand macrovascular-like endothelial cells were seen almost exclusively in the Gimap5 mutant mouse. CECs were defined by high expression levels of Cd34, Col4a2, and Sparc. Macrovascular-like cells were annotated based on the absence of LSEC (Clec4g and Dnase 113) and lymphatic (Ccl21a, Prss23, Ifi2712a, and Timp2a) gene markers and the expression of liver macrovascular endothelial markers (Plac8, Rbp1, and low expression of Rspo3). scRNA-seq data have been deposited in the National Center for Biotechnology In-formation's Gene Expression Omnibus (accession no. GSE158988). The mouse Gimap5-dependent liver endothelial cell atlas (scRNA-seq data) can be interactively explored at https://cellbrowser. yalespace.org/gimap5_res0-3/?ds=gimap5.

### GSEA

GSEA software version 4.0.3 (https://www.gsea-msigdb.org/gsea/index.jsp) was used to determine whether a predefined set of genes is enriched in the observed gene expression profile. Genes were ranked according to the fold change of gene expression. Given a predefined gene set, an enrichment score(ES) is calculated to measure the overrepresentation of members of that gene set appearing at the extremes (top or bottom) of the ranked gene list. The ES is then evaluated for significance using gene-based permutation tests (1,000). The ES, in addition to the permutation P value, indicates the degree to which the defined gene set is enriched in the gene expression data. The gene sets included the preranked list of genes differentially expressed among Gimap5-deficient and sufficient liver endothelial cells as compared with a background list of mouse Gata4-dependent liver endothelial regulated genes.

### Pseudotime Trajectory Analysis

Monocle3 in R was used to conduct the analysis. LSECs, CECs, and macrovascular-like cells were subset from the larger dataset in order to perform pseudotime analysis. A dimensionality value of 20 was used for dimension reduction. Protein localization and transmembrane domain prediction Interpro 83.0 was used to predict trans-membrane domains as well as protein localization using signal sequences. Gimap5 protein sequences for both mice and humans were uploaded and analyzed using SignalP for signal sequences and THMM for transmembrane domains.

Clinical Features and Liver Biopsy Findings in Patients with Recessive Mutations in GIMAP5: (Kindred 1)

Subject P1-1 is a 25-yr-old male who is the offspring of a first cousin union. He was initially evaluated at 13 yr of age for ecchymosis. His exam was remarkable for short stature (below the third percentile), low weight (˜10th percentile), and splenomegaly. His laboratory tests revealed pancytopenia and mild elevation of transaminases. Abdominal ultrasound showed heterogeneous liver parenchyma, portosystemic collateral vasculature, and splenomegaly. Upper endoscopy showed large esophageal varices, and the patient underwent shunt surgery at 17 yr of age with improvement in esophageal varices and other collaterals. His most recent blood work revealed direct hyper-bilirubinemia and worsening coagulopathy consistent with progression of underlying liver disease.

His sister is subject P1-2, who is currently 23 yr old. She had no significant past medical history until 11 yr of age, when she presented with hemoptysis in the setting of presumed pneumonia. Like her brother, she had splenomegaly on exam. Her laboratory test results were remarkable for leukopenia, thrombocytopenia, and mildly elevated transaminases. Esophageal and gastric fundic varices were seen on endoscopy.

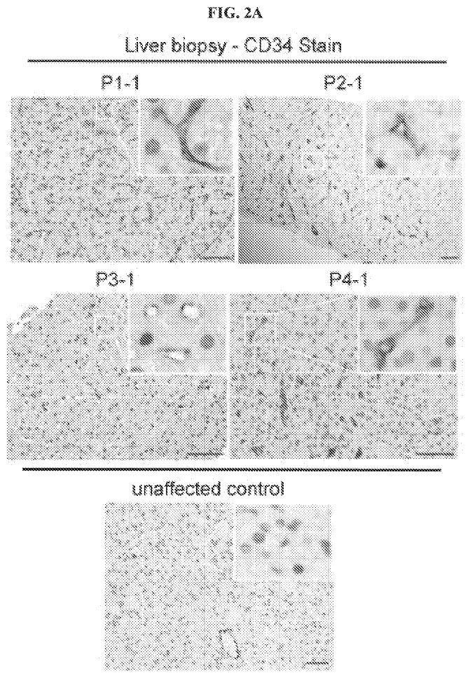

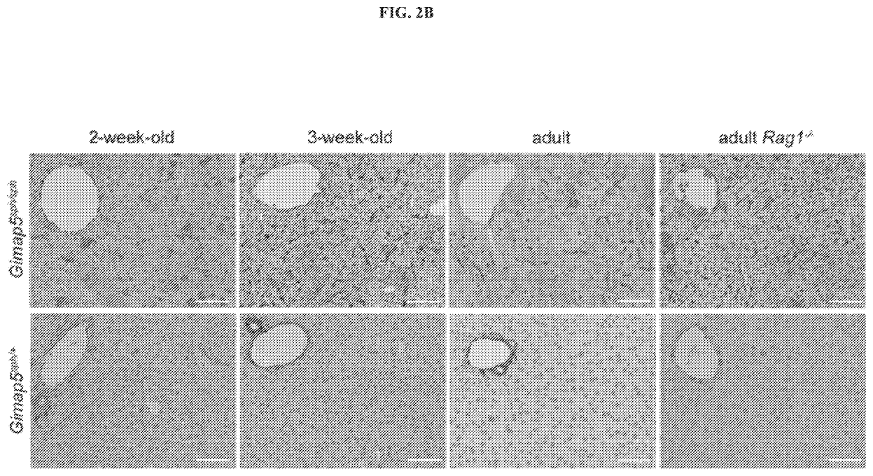

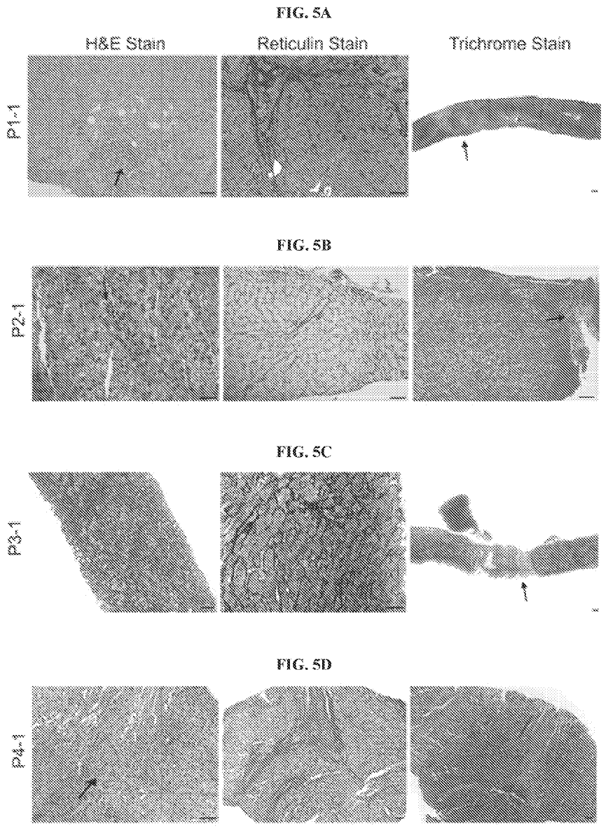

Liver biopsies of both siblings were remarkable for portal venous abnormalities consistent with idiopathic portal hypertension (FIG. 2A and FIGS. 5A-5E). Specifically, the liver biopsy from P1-1 showed heterogeneity in the architecture and scarring. One aspect of the biopsy, which could be the subcapsular tissue, showed more scarring with few fibrous septa leading to vague nodularity, while the other aspect of the biopsy showed minimal scarring and no well-formed fibrous septa. The portal venules in these fibrous septa either were not identified or showed multiple dilated channels at the periphery of the portal tracts, with some of them extending into the sinusoids. The hepatic cord pattern was largely preserved, and there were no features to suggest cirrhosis. The fibrous septa and lobules showed very minimal and focal lymphocytic infiltrates (FIGS. 5A-5E).

CD34 immunostaining showed increased staining in the sinusoidal endothelium suggesting capillarization of the sinusoids, which extended up to zone 2 in many of the lobules (FIG. 2A). The lobular parenchyma was devoid of any steatosis, hepatocytic ballooning, or acidophil necrosis. Focal and mild bile ductular proliferation was noted. Taken together, the findings are suggestive of incomplete septal cirrhosis and consistent with non-cirrhotic portal hypertension. The biopsy of subject P1-2 consisted of a scant amount of liver tissue containing only few portal areas and was fragmented, making the interpretation difficult. However, there was no evidence of cirrhosis, and no well-formed fibrous septa were noted. The portal venules were not identifiable in the portal areas. There was no significant portal or lobular inflammation, and findings in the hepatic lobules were similar to those of subject P1-1.

Clinical Features and Liver Biopsy Findings in Patients with Recessive Mutations in GIMAP5: (Kindred 2)

Subject P2-1, who died at 17 yr of age, is the offspring of a second cousin union and had recurrent episodes of fever of unknown etiology in early childhood. He subsequently developed splenomegaly and pancytopenia. Imaging studies revealed heterogeneous hepatic parenchyma and regenerative nodules, splenomegaly, and multiple para-aortocaval lymph nodes. Upper endoscopy showed esophageal varices consistent with portal hypertension. This patient's liver biopsy showed hepatic parenchyma with subtle nodularity. There is no portal inflammation, but only a few small portal areas are seen. Occasional foci of lobular inflammation are seen. The reticulin stain highlights the nodularity with zones of widened two-cell-thick plates bounded by a narrow, compressed cell plate consistent with nodular regenerative hyperplasia (FIGS. 5A-5E).

CD34 stain is positive in ˜50% of sinusoids, consistent with abnormal portal venous blood flow (FIG. 2A). The trichrome stain suggests periportal fibrosis, but there is no evidence of cirrhosis (FIGS. 5A-5E). The patient ultimately died from hypoxemic respiratory failure in the setting of Acinetobacter sepsis, Aspergillus infection, and a pulmonary embolus.

His brother, subject P2-2, presented with fatigue and epistaxis at age 15 yr and died at 22 yr of age from liver failure and portal hypertension complications, including hyperbilirubinemia, ascites, and encephalopathy. Initially, he was found to have anemia, thrombocytopenia, and splenomegaly. He experienced multiple episodes of autoimmune hemolytic anemia with a positive Coombs test result. Autoantibodies associated with liver-related autoimmunity (antinuclear antibodies, antimitochondrial antibodies, anti-liver kidney microsome and anti-smooth muscle antibodies) were absent. Over time, he showed elevated transaminases, hyperbilirubinemia, and large esophageal varices and portal hypertensive gastropathy.

His sister, subject P2-3, is a 33-yr-old female with no significant medical history until 17 yr of age, when she presented with fever, chills, and abdominal/back pain and was found to have splenomegaly. At 22 yr of age, she had an episode of bleeding in the setting of severe thrombocytopenia, which improved after i.v. Ig and corticosteroid treatment. She is currently asymptomatic.

His other sister, subject P2-4, is a 31-yr-old female with splenomegaly and diffusely coarse liver parenchyma with a lobulated contour seen on imaging studies. She is currently asymptomatic. The maternal uncle of this sibship, subject P2-5, died at 44 yr of age from infection and liver complications. He was asymptomatic until 35 yr of age, when he presented with fatigue. He was found to have easy bruising, epistaxis, gingival bleeding, splenomegaly, thrombocytopenia, and elevated transaminases of unknown etiology. He subsequently developed jaundice, pancytopenia, and dyspnea. Contrast-enhanced abdominal imaging revealed splenomegaly and collateral circulation consistent with portal hypertension, coarse granular liver parenchyma, and one 2-cm hepatic lesion with arterial enhancement and portal venous washout highly suspicious for hepatocellular carcinoma.

Clinical Features and Liver Biopsy Findings in Patients with Recessive Mutations in GIMAP5: (Kindred 3)

Subject P3-1 is a 7-yr-old male who is the offspring of a first cousin union. He was found to have hepatosplenomegaly at 2 yr of age. His laboratory tests were remarkable for pancytopenia and elevation of liver transaminases and γ-glutamyl transferase. His liver biopsy was small (barely 1 cm) with only three portal areas. The liver biopsy showed largely preserved architecture. There was one area of hepatocellular collapse that was devoid of any hemorrhage, inflammation, or scarring. Portal tracts appeared normal on a low-magnification image, except one enlarged portal area with fibrosis. On closer examination, the portal tract showed a sclerotic, narrow-caliber portal venule and multiple small venules. The reticulin stain showed collapse of reticulin framework in areas of hepatocyte loss, and the cord architecture was preserved in other areas (FIGS. 5A-5E). The CD34 stain showed increased sinusoidal endothelial staining, particularly near the portal tracts, with a heterogeneous staining pattern in the hepatic lobules (FIGS. 2A-2B). Abdominal ultrasound revealed ascites. Upper endoscopy showed esophageal varices.

Clinical Features and Liver Biopsy Findings in Patients with Recessive Mutations in GIMAP5: (Kindred 4)

Subject P4-1 is a 21-yr-old male who presented at 14 yr of age with petechia. Per an initial report, this patient suffered from recurrent thrombocytopenia, mild hemolytic anemia, neutropenia, and lymphopenia. The patient was given a working diagnosis of immunodeficiency with features of autoimmunity. Ultimately, he was diagnosed with GIMAP5 deficiency at 16 yr of age. Recently, at the age of 21, he was admitted with worsening jaundice, pancytopenia, and splenomegaly and was found to have noncirrhotic portal hypertension. His liver biopsy was remarkable for incomplete fibrous septa and features of nodular regenerative hyperplasia (FIGS. 5A-5E).

Like patients P1-1, P2-1, and P3-1, this subject's liver tissue revealed abnormal sinusoidal CD34 ex-pression (FIG. 2A). Computed tomographic angiography of the abdomen and magnetic resonance imaging per hepatoma protocol confirmed portal hypertension with splenorenal shunt and esophageal varices and showed no evidence of portal or hepatic vein thrombosis. Upper endoscopy confirmed esophageal varices.

Subject P4-1 was the only individual in this cohort who underwent a direct portal venogram. He was found to have an increased portal pressure of 14 mm Hg (normal portal venous pressure ranges between 5 and 10 mm Hg). The patient underwent recent elective splenectomy for worsening splenomegaly and thrombocytopenia and is clinically stable with close outpatient follow-up.

### Example 1: Association of Rare Damaging Recessive Genotypes in GIMAP5 and Portal Hypertension

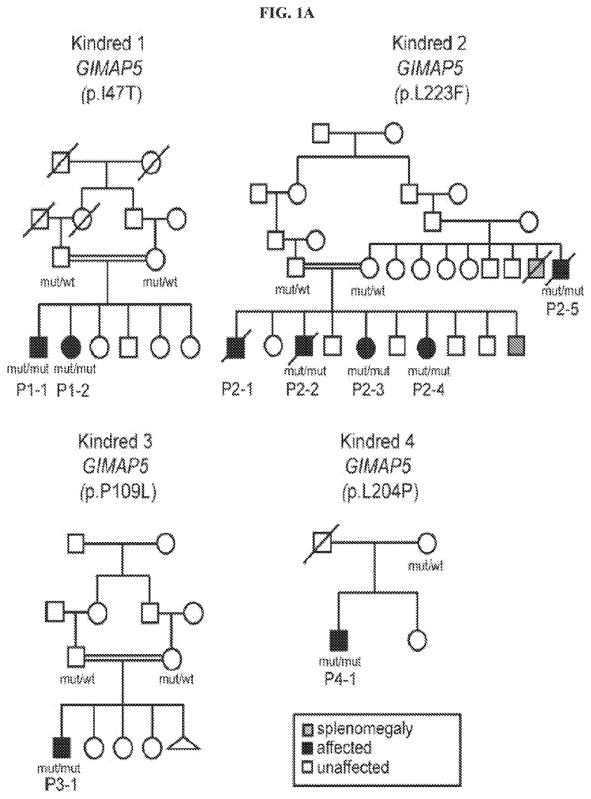

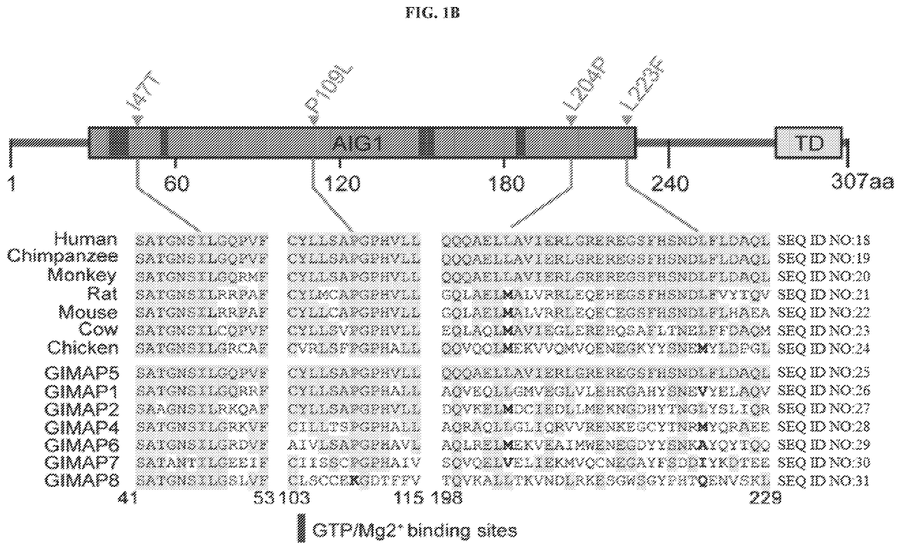

Nine individuals from four unrelated families with unexplained portal hypertension were identified (FIGS. 1A-1B). All affected subjects have splenomegaly; thrombocytopenia, elevated transaminases, and esophageal varices were nearly universal (Tables 1-2). Esophageal varices, a consequence of increased portal pressure, were confirmed in seven subjects by endoscopy. None had cirrhosis, splanchnic venous thrombosis, or other extrahepatic causes of portal hypertension.

TABLE 1
Summary of demographics, clinical presentation, genotype,
and liver features of patients with GIMAP5 deficiency

| | Age at | Current | | | | |
|---|---|---|---|---|---|---|
| | presentation | age | | Genotype | AST | ALT |
| Patient | (yr) | (yr) | Clinical presentation | GIMAP5 | (U/L) | (U/L) |
| P1-1 | 13 | 25 | Ecchymosis | p.I47T | 65 | 53 |
| P1-2 | 11 | 23 | Hemoptysis | p.I47T | 86 | 70 |
| P2-1 | 2 | 17^{a} | Recurrent infections | n/a | 49 | 54 |
| P2-2 | 15 | 22^{a} | Fatigue, epistaxis | p.L223F | 85 | 247 |
| P2-3 | 17 | 33 | Abdominal and back pain | p.L223F | n/a | n/a |
| P2-4 | n/a | 31 | Recognized during screening | p.L223F | n/a | n/a |
| | | | of family members | | | |
| P2-5 | 35 | 44^{a} | Fatigue | p.L223F | 40 | 37 |
| P3-1 | 2 | 7 | Elevated transaminases and | p.P109L | 113 | 76 |
| | | | GGT, and | | | |
| | | | hepatosplenomegaly | | | |
| P4-1 | 14 | 21 | Petechia | p.L204P | 40 | 20 |

^{a}Age at death

TABLE 2
Portal hypertension and hepatocellular carcinoma
in patients with GIMAP5 deficiency

| | | | Esophageal | | Hepatocellular |
|---|---|---|---|---|---|
| Patient | Splenomegaly | Thrombocytopenia | varices | Ascites | carcinoma |
| P1-1 | Yes | Yes | Yes | No | No |
| P1-2 | Yes | Yes | Yes | No | No |
| P2-1 | Yes | Yes | Yes | No | No |
| P2-2 | Yes | Yes | Yes | Yes | No |
| P2-3 | Yes | Yes | n/a | No | No |
| P2-4 | Yes | No | n/a | No | No |
| P2-5 | Yes | Yes | Yes | No | Yes |
| P3-1 | Yes | Yes | Yes | Yes | No |
| P4-1 | Yes | Yes | Yes | No | No |

Notably, three affected individuals in kindred 2 are deceased, two of which died from complications of portal hypertension. Seven affected individuals from these families were subjected to whole-exome sequencing to provide high depth of coverage across all coding bases and flanking intronic segments, and variants were called. Variants of interest were genotyped by sequencing of PCR amplicons in all other available kindred members. Because affected members of three of the four families are consanguineous and none of the parents of affected subjects are themselves affected (FIGS. 1A-1B), potential causative variants were sought under a recessive model of transmission.

All available affected members of these four kindreds shared different rare homozygous missense variants in the gene GIMAP5 predicted to be damaging by Polyphen-2 and had Combined Annotation Dependent Depletion scores >20 (FIGS. 1A-1B). GIMAP5 encodes GTPase of the immunity-associated protein (GIMAP) family member 5, a small GTPase predominantly expressed in lymphocytes that regulates lymphocyte survival.

In kindred 1, two affected siblings, the offspring of a first cousin union, were homozygous for a variant encoding a p.147T substitution (FIGS. 1A-1B). The I47T variant has an allele frequency of zero in gnomAD.

In kindred 2, there were four affected offspring of a second cousin union and an additional affected subject who is a sibling of one of the unaffected parents of this sibship. Four affected subjects were homozygous for the p.L223F variant, which also has an allele frequency of 0 in gnomAD. The genotype of P2-1 could not be confirmed, as he was already deceased.

In kindred 3, a child of a first cousin union with noncirrhotic portal hypertension was homozygous for a p.P109L substitution. This variant allele is extremely rare, seen once among more than 251,400 alleles sequenced from diverse populations (allele frequency in gnomAD of 3.982× 10-6). Subject P4-1 is a 21-yr-old male with newly recognized noncirrhotic portal hypertension. He is the sole affected offspring of unrelated parents and has been previously reported with hemolytic anemia, thrombocytopenia, lymphopenia, splenomegaly, and GIMAP5 deficiency. He is homozygous for a p.L204P substitution that has an allele frequency of 2.1 ×10³ (FIGS. 1A-1B and Tables 1-2). Notably, all four mutations (p.I47T, p.P109L, p.L204P, and p.L223F) lead to loss of GIMAP5 protein expression.

The cosegregation of rare damaging homozygous genotypes that produce nonconservative substitutions at highly conserved amino acids with noncirrhotic portal hypertension is unlikely to have occurred by chance. The probands of these kindreds each had only one to nine rare damaging homozygous genotypes, and no other gene with a homozygous damaging genotype was shared among all kindreds. These variants were rare in all populations examined.

Parametric analysis of linkage in the three consanguineous kindreds specifying damaging GIMAP5 variants as rare, with very high penetrance of portal hypertension via recessive genotypes and very rare phenocopies, was performed. Logarithm of the odds scores in favor of linkage at a recombination fraction of 0 in kindreds 1-3 were, 1.8, >3.3 (the likelihood of the second allele in subject P2-6 being inherited by chance is indeterminate, and the genotype of deceased P2-1 could not be confirmed), and 1.2, respectively, providing a combined logarithm of the odds score >6.3 (odds 2 million to 1 in favor of linkage). Therefore, strong statistical support for the role of these rare recessive GIMAP5 genotypes in noncirrhotic portal hypertension was observed.

Collectively, the identification of four families with independent rare damaging recessive genotypes in GIMAP5 that precisely cosegregate within families affected by noncirrhotic portal hypertension provides strong evidence that these genotypes cause portal hypertension in these families.

### Example 2: Role of Gimap5 Deficiency in Sinusoidal Endothelium in Liver Disease and Portal Hypertension

Liver biopsies were obtained from both affected siblings of kindred 1, P2-1 of kindred 2, and affected individuals in kindreds 3 and 4. Liver parenchyma from subjects P1-1 and P2-1 showed features consistent with nodular regenerative hyperplasia (FIGS. 5A-5E). Importantly, marked CD34 immunostaining in the LSECs was found which is consistent with capillarization (FIG. 2A).

As observed in her siblings biopsy, the liver parenchyma of P1-2 lacked significant fibrosis or septa formation, and venules were absent in the portal areas, features consistent with idiopathic noncirrhotic portal hypertension. Liver biopsy from subject P3-1 was small and showed no cirrhosis with a largely preserved architecture. This sample also showed increased CD34 staining in LSECs (FIG. 2A), particularly near the portal tracts, with a heterogeneous staining pattern in the hepatic lobules. In contrast, in unaffected healthy individuals, CD34 is solely expressed in macrovascular hepatic vessels and absent on LSECs (FIG. 2A).

Subject P4-1's liver biopsy showed features of nodular regenerative hyperplasia, no cirrhosis, and aberrant CD34 sinusoidal stain (FIG. 2A and FIGS. 5A-5E). This patient underwent direct portal venography, which showed increased portal pressure to 14 mm Hg (normal portal venous pressure, 5-10 mm Hg).

The liver abnormalities seen in these patients are reproduced in two independent GIMAP5-deficient mouse models. Both strains showed splenomegaly, nodular regenerative hyperplasia of the liver, and decreased life expectancy. This distinctive finding provides strong evidence that the recessive GIMAP5 genotypes found in humans result in loss of normal GIMAP5 function.

Since liver disease and portal hypertension can be found in other immunodeficiencies and autoimmune syndromes, it was examined whether the liver phenotype in GIMAP5 loss-of-function (LOF) mutant mice (i.e., Gimap5sph/sph) is a consequence of underlying defects in adaptive immune cells. Gimap5sph/sph mice were crossed with Rag1−/−mice, which lack B and T cells and do not have evidence of portal hypertension, to produce mice homozygous for both Gimap5 LOF mutation and Rag1 deficiency.

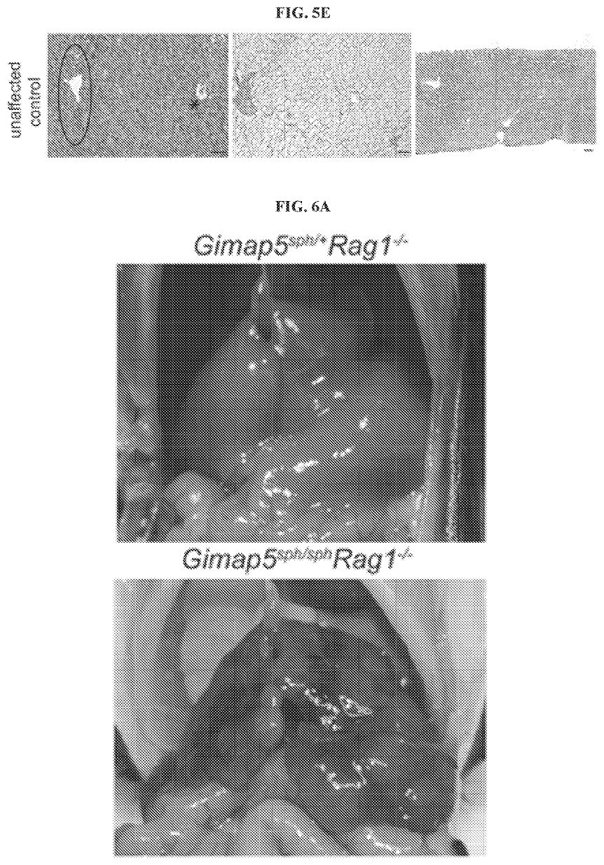

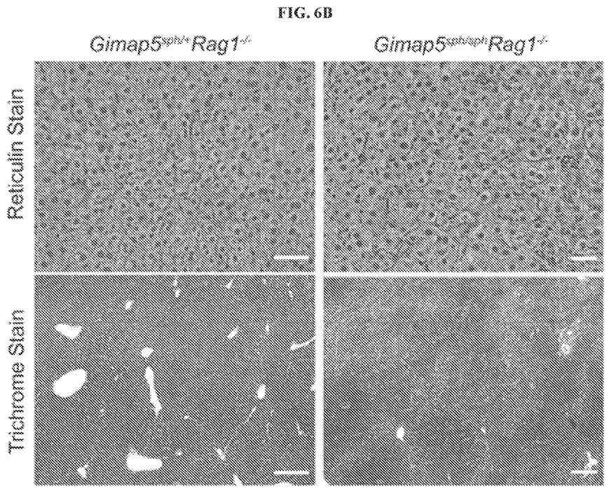

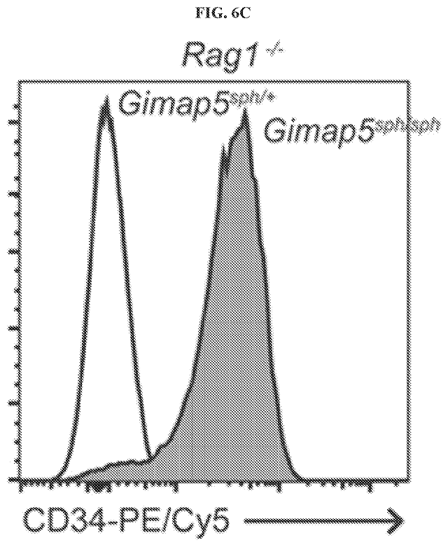

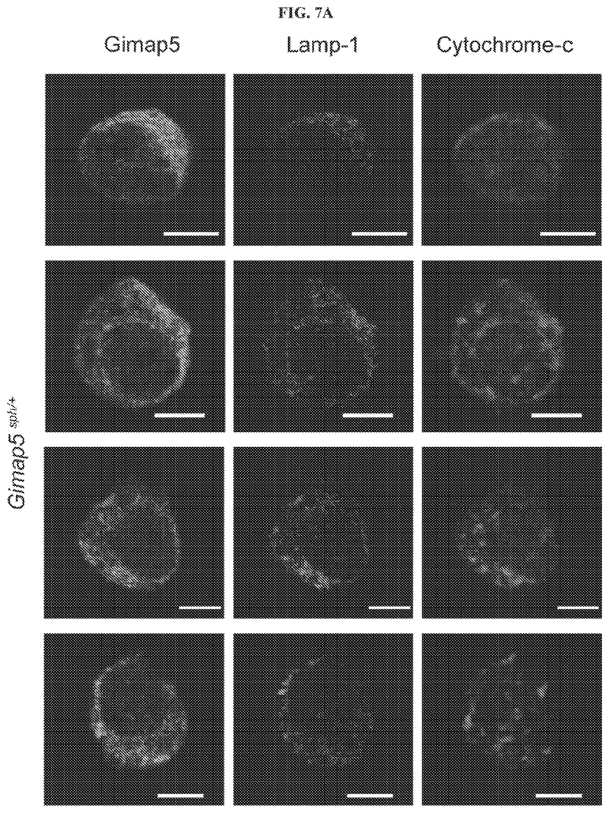

These mice showed the same liver abnormalities seen in Gimap5sph/sph mice (FIGS. 6A-6C), indicating that the liver disease is not caused by aberrant B or T cell function. This result is consistent with prior reports showing that transplantation of Gimap5-deficient bone marrow cells into lethally irradiated WT recipient mice did not induce liver pathology.

Further examination of the livers of Gimap5sph/sph mutant mice showed aberrant expression of CD34 in sinusoidal endothelium, as seen in GIMAP5 mutant patients. CD34 overexpression could be detected as early as 2 weeks of age in these mice and in mice with both Gimap5 and Rag1 deficiency (FIG. 2B and FIGS. 6A-6C). Accordingly, it was hypothesized that Gimap5 deficiency in sinusoidal endothelium may be important for liver disease and portal hypertension.

### Example 3: Gimap5 Expression in Liver Endothelial Cells is Critical for Preserving Normal LSEC Specification and Identity

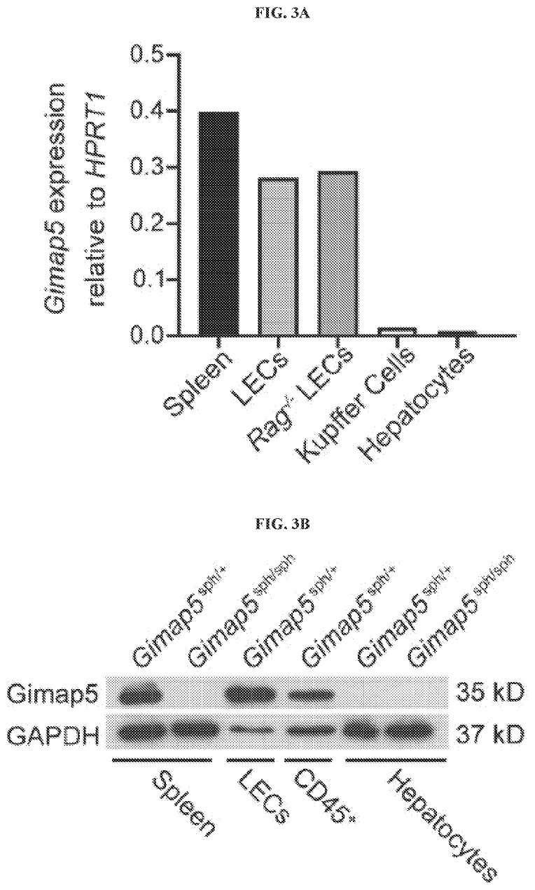

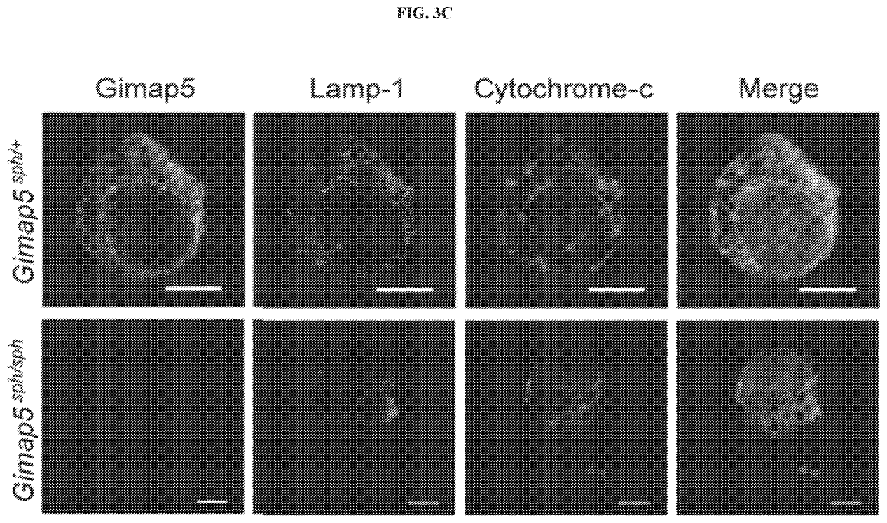

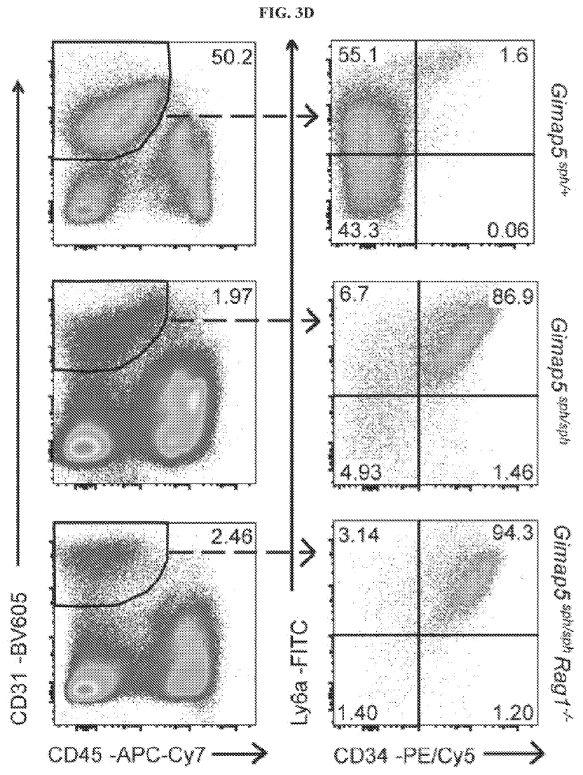

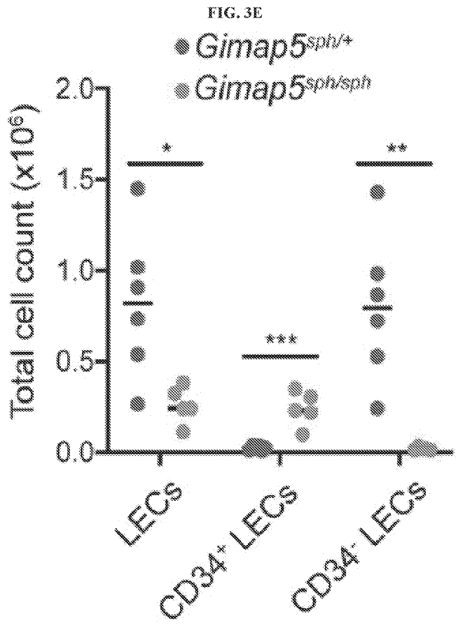

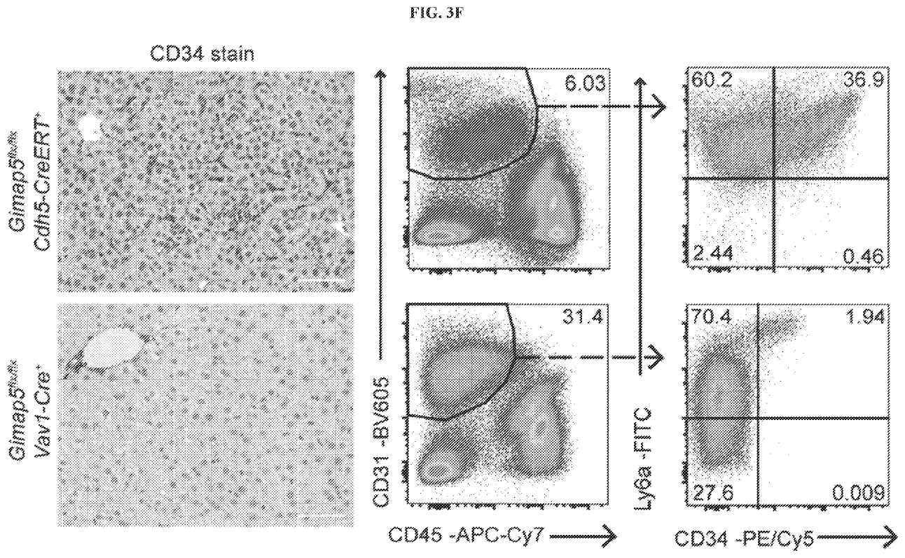

Because GIMAP5 is reported to be primarily expressed in lymphocytes, it was examined whether Gimap5 is expressed in the liver and, if so, in which cell types. We found that CD45-CD31+ liver endothelial cells, but not hepatocytes, express Gimap5 mRNA and protein (FIGS. 3A-3B). Analysis of publicly available single-cell RNA sequencing (scRNA-seq) from mouse and human healthy livers confirmed Gimap5 gene expression in hepatic endothelial cells.

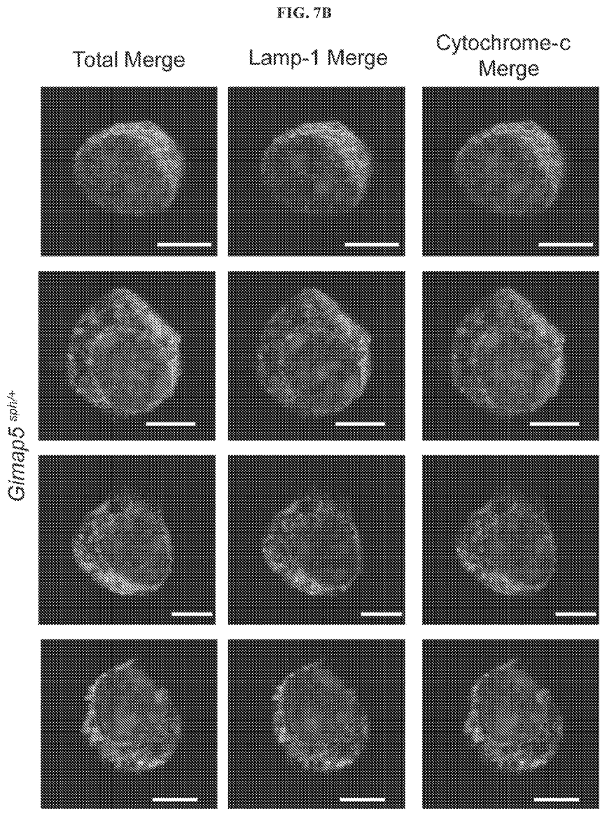

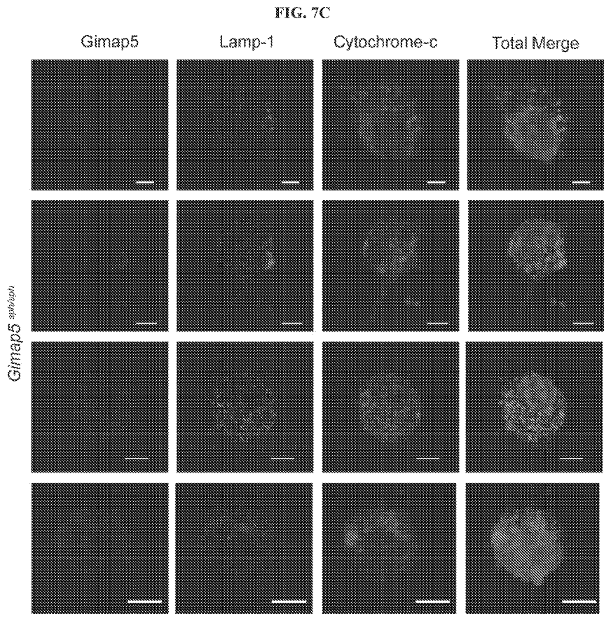

Subsequently, the subcellular localization of Gimap5 in CD45-CD31+ liver endothelial cells was examined using confocal microscopy. It was found that Gimap5 protein colocalizes with lysosomal marker Lamp1, supporting its localization within the lysosomes and associated compartments, although some signal could also be detected in the cytosol (FIG. 3C and FIGS. 7A-7C). This finding is consistent with studies using a monoclonal antibody against Gimap5 in Jurkat T cells, in CD4+ Tcells, and overexpression systems using lower plasmid amounts. Bioinformatic studies did not identify any signal peptides, but it was predicted a type 4 transmembrane domain that would support Gimap5 localization to the lysosomal membrane.

Liver endothelial cells isolated from Gimap5sph/sph mutant mice in both WT and Rag1 deficient backgrounds showed markedly increased CD34 expression by flow cytometry as compared with hepatic endothelial cells from control mice (FIG. 3D). Moreover, an approximately three-fold reduction in the absolute number of CD45-CD31+ endothelial cells was detected in Gimap5sph/sph livers as compared with heterozygous mice, and most of these cells express CD34 in contrast to low or absent CD34 staining in control mice (FIG. 3E). These observations strongly suggest that intrinsic Gimap5 expression in liver endothelial cells is critical for preserving normal LSEC specification and identity, which in turn are critical for normal liver function.

To further test this conclusion, Gimap5flx/flx mice were crossed with Cdh5 (PAC)-CreERT2 mice or with Vav1-Cre mice. These approaches eliminate Gimap5 expression exclusively in endothelial cells or hematopoietic cells, respectively. It was found that tamoxifen-induced Gimap5 deletion in endothelial cells led to aberrant liver sinusoidal CD34 expression, whereas constitutive Gimap5 deletion in Vav1+ hematopoietic cells caused no detectable liver abnormalities or abnormal CD34 expression in LSECs (FIG. 3F).

These findings revealed that the liver phenotype seen in Gimap5sph/sph mice results primarily from the lack of GIMAP5 in liver endothelial cells and is not the result of dysregulated GIMAP5-deficient immune cells.

### Example 4: GIMAP5 as a Critical Regulator of Liver Endothelial Cell Homeostasis

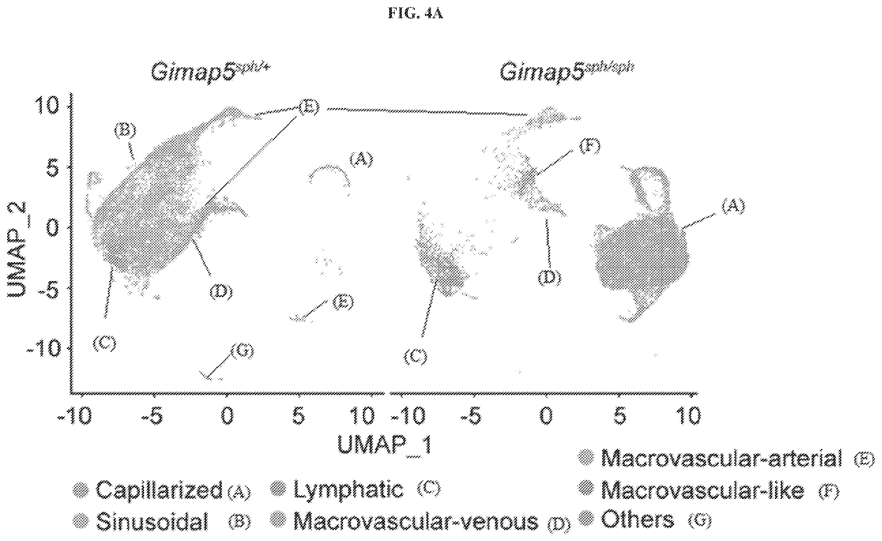

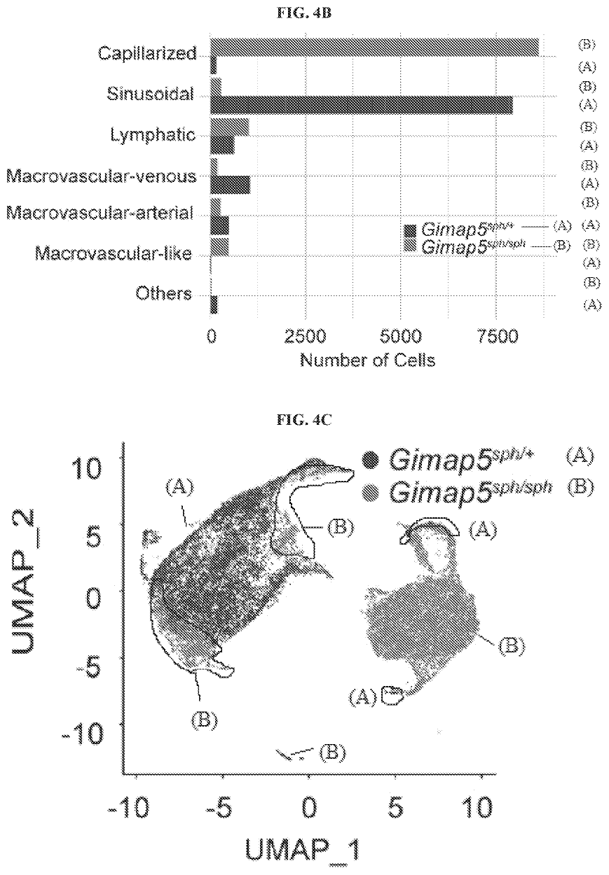

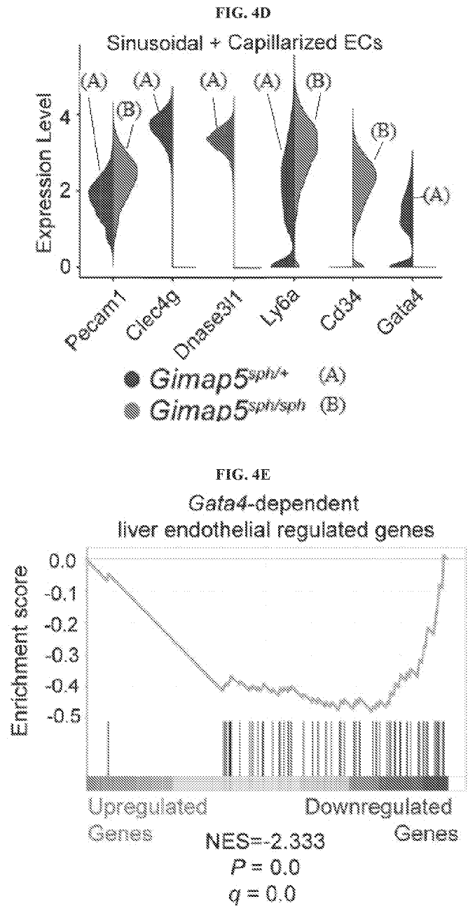

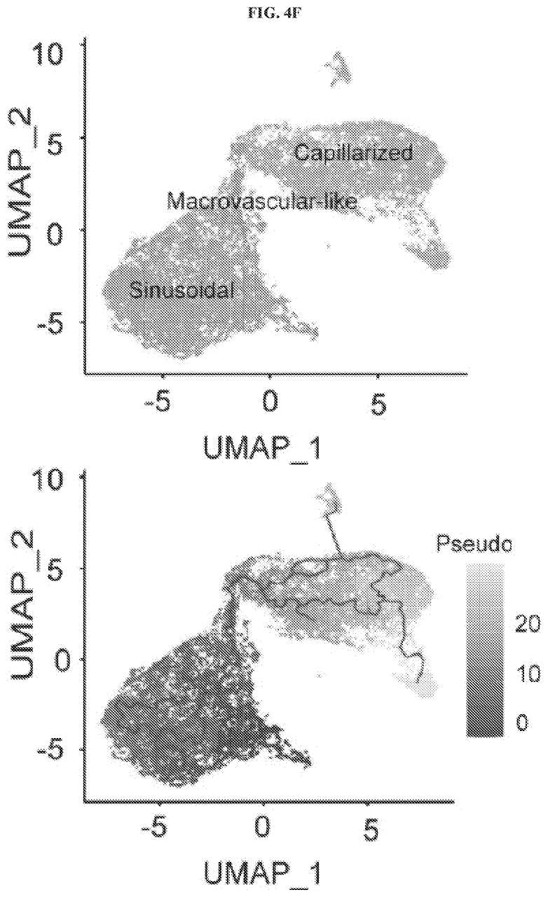

Given the important role of Gimap5 in liver endothelial cells, scRNA-seq of CD45-CD31+ liver endothelial cells from mice that were either sufficient or deficient for Gimap5 was performed. Clustering of the results identified clearly distinct populations, with the vast majority representing either LSECs or CECs (FIGS. 4A-4B). Remarkably, the Gimap5sph/sph liver showed a near absence of LSECs and the emergence of a CEC population, a reduction in macrovascular venous and macrovascular arterial endothelial cells, and an expansion of lymphatic endothelial cells (FIGS. 4A-4B).

CECs present in GIMAP5-deficient liver were notable for the absence of GATA4 expression, a transcription factor required for LSEC specification that suppresses expression of genes required for capillarization of endothelial cells. CECs also showed an absence of classical LSEC markers (e.g., Clec4g, Dnase311), overexpression of markers associated with capillarized endothelium (e.g., CD34, Pecam1; FIGS. 4C-4D), and up-regulation of genes involved in extracellular matrix organization (e.g., Col4a2 and Sparc).

Gene set enrichment analysis (GSEA) showed that GIMAP5-deficient CD45-CD31+ cells had markedly lower expression of genes that are Gata4 dependent in LSECs (FIG. 4E). Collectively, these findings place GIMAP5 upstream of the LSEC-specifying GATA4 transcription factor. These data are consistent with previous observations that GATA4 expression was significantly decreased in human livers with advanced liver fibrosis and cirrhosis, a setting in which endothelial cell capillarization is commonly observed. Notably, in addition to the expansion of CECs, Gimap5 deficiency also revealed the emergence of a macrovascular-like cell type that is not seen under homeostatic conditions (FIGS. 4A-4B).

To better understand the relationship between these cells, LSECs and CECs, we performed pseudotime trajectory analysis. This analysis suggests that LSECs transition into CECs via these macrovascular-like cells (FIG. 4F).

In several primary immunodeficiency syndromes, it has been proposed that accompanying liver disease results from hyper-inflammation caused by a dysregulated immune system. The present disclosure demonstrates that liver abnormalities in GIMAP5 deficiency are independent of immune cell function and result from liver endothelial cell dysfunction. These findings are consistent with the possibility that nodular regenerative hyperplasia of the liver is primarily caused by a sinusoidal injury.

This study identifies GIMAP5 as a critical regulator of liver endothelial cell homeostasis, and, when absent, it produces portal hypertension. Liver endothelial cells, and in particular LSECs, are essential in regulating the hepatic vascular tone and maintenance of a regulated low portal pressure. Under pathological conditions, such as liver fibrosis, LSECs de-differentiate into CECs.

It is presently unknown whether or how GIMAP5 activity is normally regulated and whether this regulation plays a continuous role in modulating function of LSECs. For example, without wishing to be bound by theory, it may change the degree of fenestration or synthesis of basement membrane, both characteristics of CECs. It is also possible that Gimap5 functions to promote the stability of pro-survival signals, such as Mcl-1, besides controlling GATA4 expression and LSEC homeostasis. Since GIMAP5 is expressed in liver endothelial cells and not exclusively in LSECs, and because Gimap5 deficiency results in a reduction in macrovascular endothelial cells in addition to a nearly complete replacement of LSECs by CECs, further studies are required to determine the individual contributions of GIMAP5-expressing endothelial cell subsets to the liver abnormalities seen in both Gimap5-deficient mice and humans.

Without wishing to be bound by theory, it may be the case that GIMAP5-mediated signal transduction events are continuously required for the maintenance of LSEC identity. It will be of interest to determine whether signals resulting from normal or diseased liver alter GIMAP5 activity to promote or inhibit local perfusion via regulation of LSEC identity. This understanding might provide a means to prevent or mitigate development of portal hypertension in patients with advanced liver disease.

This study provides new cellular and molecular insights into the pathogenesis of portal hypertension, an important step toward uncovering new targeted therapeutic agents. Thus, genetic investigation provides new insights into severe liver disease and novel treatment approaches.

Sequence Listing
GIMAP5 (homo sapiens)
SEQ ID NO: 1
MGGFQRGKYGTMAEGRSEDNLSATPPALRIILVGKTGCGKSATGNSILGQPVFESKLRAQSVTR
TCQVKTGTWNGRKVLVVDTPSIFESQADTQELYKNIGDCYLLSAPGPHVLLLVIQLGRFTAQDT
VAIRKVKEVFGTGAMRHVVILFTHKEDLGGQALDDYVANTDNCSLKDLVRECERRYCAFNNWGS
VEEQRQQQAELLAVIERLGREREGSFHSNDLFLDAQLLQRTGAGACQEDYRQYQAKVEWQVEKH
KQELRENESNWAYKALLRVKHLMLLHYEIFVFLLLCSILFFIIFLFIFHYI
GATA4 (homo sapiens)
SEQ ID NO: 2
MYQSLAMAANHGPPPGAYEAGGPGAFMHGAGAASSPVYVPTPRVPSSVLGLSYLQGGGAG
SASGGASGGSSGGAASGAGPGTQQGSPGWSQAGADGAAYTPPPVSPRFSFPGTTGSLAAA
AAAAAAREAAAYSSGGGAAGAGLAGREQYGRAGFAGSYSSPYPAYMADVGASWAAAAAAS
AGPFDSPVLHSLPGRANPAARHPNLDMFDDFSEGRECVNCGAMSTPLWRRDGTGHYLCNA
CGLYHKMNGINRPLIKPQRRLSASRRVGLSCANCQTTTTTLWRRNAEGEPVCNACGLYMK
LHGVPRPLAMRKEGIQTRKRKPKNLNKSKTPAAPSGSESLPPASGASSNSSNATTSSSEE
MRPIKTEPGLSSHYGHSSSVSQTFSVSAMSGHGPSIHPVLSALKLSPQGYASPVSQSPQT
SSKQDSWNSLVLADSHGDIITA
MAF (homo sapiens)
SEQ ID NO: 3
MASELAMSNSDLPTSPLAMEYVNDFDLMKFEVKKEPVETDRIISQCGRLIAGGSLSSTPM
STPCSSVPPSPSFSAPSPGSGSEQKAHLEDYYWMTGYPQQLNPEALGFSPEDAVEALISN
SHQLQGGFDGYARGAQQLAAAAGAGAGASLGGSGEEMGPAAAVVSAVIAAAAAQSGAGPH
YHHHHHHAAGHHHHPTAGAPGAAGSAAASAGGAGGAGGGGPASAGGGGGGGGGGGGGGAA
GAGGALHPHHAAGGLHFDDRFSDEQLVTMSVRELNRQLRGVSKEEVIRLKQKRRTLKNRG
YAQSCRFKRVQQRHVLESEKNQLLQQVDHLKQEISRLVRERDAYKEKYEKLVSSGFRENG
SSSDNPSSPEFFM
MEIS2 (homo sapiens)
SEQ ID NO: 4
MAQRYDELPHYGGMDGVGVPASMYGDPHAPRPIPPVHHLNHGPPLHATQHYGAHAPHPNV
MPASMGSAVNDALKRDKDAIYGHPLFPLLALVFEKCELATCTPREPGVAGGDVCSSDSFN
EDIAVFAKQVRAEKPLFSSNPELDNLMIQAIQVLRFHLLELEKVHELCDNFCHRYISCLK
GKMPIDLVIDERDGSSKSDHEELSGSSTNLADHNPSSWRDHDDATSTHSAGTPGPSSGGH
ASQSGDNSSEQGDGLDNSVASPGTGDDDDPDKDKKRQKKRGIFPKVATNIMRAWLFQHLT
HPYPSEEQKKQLAQDTGLTILQVNNWFINARRRIVQPMIDQSNRAGFLLDPSVSQGAAYS
PEGQPMGSFVLDGQQHMGIRPAGLQSMPGDYVSQGGPMGMSMAQPSYTPPQMTPHPTQLR
HGPPMHSYLPSHPHHPAMMMHGGPPTHPGMTMSAQSPTMLNSVDPNVGGQVMDIHAQ
PDGFβ (homo sapiens)
SEQ ID NO: 5
MNRCWALFLSLCCYLRLVSAEGDPIPEELYEMLSDHSIRSFDDLQRLLHGDPGEEDGAEL
DLNMTRSHSGGELESLARGRRSLGSLTIAEPAMIAECKTRTEVFEISRRLIDRTNANFLV
WPPCVEVQRCSGCCNNRNVQCRPTQVQLRPVQVRKIEIVRKKPIFKKATVTLEDHLACKC
ETVAAARPVTRSPGGSQEQRAKTPQTRVTIRTVRVRRPPKGKHRKFKHTHDKTALKETLG
A
VEGFα (homo sapiens)
SEQ ID NO: 6
MNFLLSWVHWSLALLLYLHHAKWSQAAPMAEGGGQNHHEVVKFMDVYQRSYCHPIETLVD
IFQEYPDEIEYIFKPSCVPLMRCGGCCNDEGLECVPTEESNITMQIMRIKPHQGQHIGEM
SFLQHNKCECRPKKDRARQEKKSVRGKGKGQKRKRKKSRYKSWSVYVGARCCLMPWSLPG
PHPCGPCSERRKHLFVQDPQTCKCSCKNTDSRCKARQLELNERTCRCDKPRR
APLN (homo sapiens)
SEQ ID NO: 7
MNLRLCVQALLLLWLSLTAVCGGSLMPLPDGNGLEDGNVRHLVQPRGSRNGPGPWQGGRR
KFRRQRPRLSHKGPMPF
MYC (homo sapiens)
SEQ ID NO: 8
MPLNVSFTNRNYDLDYDSVQPYFYCDEEENFYQQQQQSELQPPAPSEDIWKKFELLPTPP
LSPSRRSGLCSPSYVAVTPFSLRGDNDGGGGSFSTADQLEMVTELLGGDMVNQSFICDPD
DETFIKNIIIQDCMWSGFSAAAKLVSEKLASYQAARKDSGSPNPARGHSVCSTSSLYLQD
LSAAASECIDPSVVFPYPLNDSSSPKSCASQDSSAFSPSSDSLLSSTESSPQGSPEPLVL
HEETPPTTSSDSEEEQEDEEEIDVVSVEKRQAPGKRSESGSPSAGGHSKPPHSPLVLKRC
HVSTHQHNYAAPPSTRKDYPAAKRVKLDSVRVLRQISNNRKCTSPRSSDTEENVKRRTHN
VLERQRRNELKRSFFALRDQIPELENNEKAPKVVILKKATAYILSVQAEEQKLISEEDLL
RKRREQLKHKLEQLRNSCA
GATA6 (homo sapiens)
SEQ ID NO: 9
MALTDGGWCLPKRFGAAGADASDSRAFPAREPSTPPSPISSSSSSCSRGGERGPGGASNC
GTPQLDTEAAAGPPARSLLLSSYASHPFGAPHGPSAPGVAGPGGNLSSWEDLLLFTDLDQ
AATASKLLWSSRGAKLSPFAPEQPEEMYQTLAALSSQGPAAYDGAPGGFVHSAAAAAAAA
AAASSPVYVPTTRVGSMLPGLPYHLQGSGSGPANHAGGAGAHPGWPQASADSPPYGSGGG
AAGGGAAGPGGAGSAAAHVSARFPYSPSPPMANGAAREPGGYAAAGSGGAGGVSGGGSSL
AAMGGREPQYSSLSAARPLNGTYHHHHHHHHHHPSPYSPYVGAPLTPAWPAGPFETPVLH
SLQSRAGAPLPVPRGPSADLLEDLSESRECVNCGSIQTPLWRRDGTGHYLCNACGLYSKM
NGLSRPLIKPQKRVPSSRRLGLSCANCHTTTTTLWRRNAEGEPVCNACGLYMKLHGVPRP
LAMKKEGIQTRKRKPKNINKSKTCSGNSNNSIPMTPTSTSSNSDDCSKNTSPTTQPTASG
AGAPVMTGAGESTNPENSELKYSGQDGLYIGVSLASPAEVTSSVRPDSWCALALA
SEQ ID NO: 10
AAGATAACTTGTCTGCAACACCA
SEQ ID NO: 11
GTAGCAGTCCCCGATGTTCT
SEQ ID NO: 12
GGGGAGGACGTTCATAGCTT
SEQ ID NO: 13
TGCTCCAGGGTCCAGAGAT
SEQ ID NO: 14
GATAACTTGTCTGCAACACCA
SEQ ID NO: 15
TGTCCTGAGCAGTGAAACGC
SEQ ID NO: 16
ACATGGAACGGGAGGAAAGTC
SEQ ID NO: 17
CACGAGGATCTGAGTCTGAATTG
SEQ ID NO: 18
SATGNSILGQPVFCYLLSAPGPHVLLQQQAELLAVIERLGREREGSFHSNDLFLDAQL
SEQ ID NO: 19
SATGNSILGQPVFCYLLSAPGPHVLLQQQAELLAVIERLGREREGSFHSNDLFLDAQL
SEQ ID NO: 20
SATGNSILGQRMFCYLLSAPGPHVLLQQQAELLAVIERLGREREGSFHSNDLFLDAQL
SEQ ID NO: 21
SATGNSILRRPAFCYLMCAPGPHVLLGQLAELMALVRRLEQEHEGSFHSNDLFVYTQV
SEQ ID NO: 22
SATGNSILRRPAFCYLLCAPGPHVLLGQLAELMALVRRLEQECEGSFHSNDLFLHAEA
SEQ ID NO: 23
SATGNSILCQPVFCYLLSVPGPHVLLEQLAQLMAVIEGLEREHQSAFLTNELFFDAQM
SEQ ID NO: 24
SATGNSILGRCAFCVRLSFPGPHALLQQVQQLMEKVVQMVQENEGKYYSNEMYLDPGL
SEQ ID NO: 25
SATGNSILGQPVFCYLLSAPGPHVLLQQQAELLAVIERLGREREGSFHSNDLFLDAQL
SEQ ID NO: 26
SATGNSILGQRRFCYLLSAPGPHALLAQVEQLLGMVEGLVLEHKGAHYSNEVYELAQV
SEQ ID NO: 27
SAAGNSILRKQAFCYLLSAPGPHVLLDQVKELMDCIEDLLMEKNGDHYTNGLYSLIQR
SEQ ID NO: 28
SATGNSILGRKVFCILLTSPGPHALLAQRAQLLGLIQRVVRENKEGCYTNRMYQRAEE
SEQ ID NO: 29
SATGNSILGRDVFAIVLSAPGPHAVLAQLRELMEKVEAIMWENEGDYYSNKYAYQYTQQ
SEQ ID NO: 30
SATANTILGEEIFCIISSCPGPHAIVSQVQELVELIEKMVQCNEGAYFSDDIYKDTEE
SEQ ID NO: 31
SATGNSILGSLVFCLSCCEKGDTFFVTQVKALLTKVNDLRKESGWSGYPHTQENVSKL

### Enumerated Embodiments

The following exemplary embodiments are provided, the numbering of which is not to be construed as designating levels of importance:

Embodiment 1 provides a composition comprising one or more nucleic acid-lipid particles, wherein each nucleic acid-lipid particle comprises:
(a) a cationic lipid;
(b) a non-cationic lipid;
(c) a conjugated lipid that inhibits aggregation of two or more nucleic acid lipid particles; and
(d) a nucleic acid encapsulated within the nucleic acid-lipid particle, wherein the nucleic acid either:
   (i) encodes a protein which has a reduced abundance in a GTPase IMAP family member 5 (GIMAP5) deficient subject as compared to a healthy subject; or
   (ii) at least partially inhibits expression of a protein which has an increased abundance in a GIMAP5 deficient subject as compared to a healthy subject.

Embodiment 2 provides the composition of Embodiment 1, wherein the cationic lipid comprises about 50 mol % to about 90 mol % of the total lipid present in the nucleic acid-lipid particle.

Embodiment 3 provides the composition of Embodiment 1 or 2, wherein the non-cationic lipid is at least one selected from the group consisting of cholesterol and a phospholipid.

Embodiment 4 provides the composition of any one of Embodiments 1-3, wherein the non-cationic lipid comprises about 9.9 mol % to about 49.9 mol % of the total lipid present in the nucleic acid-lipid particle.

Embodiment 5 provides the composition of any one of Embodiments 1-4, wherein the conjugated lipid that inhibits aggregation of two or more nucleic acid-lipid particles comprises a polyethyleneglycol (PEG)-lipid conjugate.

Embodiment 6 provides the composition of any one of Embodiments 1-5, wherein the conjugated lipid comprises about 0.1 mol % to about 2 mol % of the total lipid present in the nucleic acid-lipid particle.

Embodiment 7 provides the composition of any one of Embodiments 1-6, wherein the nucleic acid encodes a protein which has a reduced abundance in a GIMAP5 deficient subject as compared to a healthy subject.

Embodiment 8 provides the composition of Embodiment 7, wherein the protein which has a reduced abundance in a GIMAP5 deficient subject as compared to a healthy subject is an enzyme.

Embodiment 9 provides the composition of Embodiment 8, wherein the enzyme is GIMAP5.

Embodiment 10 provides the composition of Embodiment 9, wherein the nucleic acid comprises a messenger RNA (mRNA) which encodes a protein that shares at least 85%, 90%, 95%, 96%, 97%, 98%, or 99% sequence homology with SEQ ID NO:1.

Embodiment 11 provides the composition of Embodiment 10, wherein the mRNA encodes SEQ ID NO: 1.

Embodiment 12 provides the composition of Embodiment 7, wherein the protein which has a reduced abundance in a GIMAP5 deficient subject as compared to a healthy subject is selected from the group consisting of GATA4, MAF, and MEIS2.

Embodiment 13 provides the composition of Embodiment 12, wherein the nucleic acid comprises a messenger RNA (mRNA) which encodes a protein that shares at least 85%, 90%, 95%, 96%, 97%, 98%, or 99% sequence homology with a sequence selected from the group consisting of SEQ ID NO:2, SEQ ID NO:3, and SEQ ID NO:4.

Embodiment 14 provides the composition of Embodiment 13, wherein the mRNA encodes a sequence selected from the group consisting of SEQ ID NO:2, SEQ ID NO:3, and SEQ ID NO:4.

Embodiment 15 provides the composition of any one of Embodiments 1-6, wherein the nucleic acid at least partially inhibits expression of a protein which has an increased abundance in a GIMAP5 deficient subject as compared to a healthy subject.

Embodiment 16 provides the composition of Embodiment 15, wherein the protein which has an increased abundance in a GIMAP5 deficient subject is selected from the group consisting of PDGFβ, VEGFα, APLN, MYC, and GATA6.

Embodiment 17 provides the composition of Embodiment 15 or 16, wherein the nucleic acid comprises a small interfering RNA (siRNA).

Embodiment 18 provides the composition of Embodiment 17, wherein the siRNA at least partially inhibits expression of a protein that shares at least 85%, 90%, 95%, 96%, 97%, 98%, or 99% sequence homology with a sequence selected from the group consisting of SEQ ID NO:5, SEQ ID NO:6, SEQ ID NO:7, SEQ ID NO:8, and SEQ ID NO:9.

Embodiment 19 provides the composition of Embodiment 17 or 18, wherein the siRNA at least partially inhibits expression of a protein with a sequence selected from the group consisting of SEQ ID NO:5, SEQ ID NO:6, SEQ ID NO:7, SEQ ID NO:8, and SEQ ID NO:9.

Embodiment 20 provides a composition comprising one or more polymer-based vehicles, wherein the polymer-based vehicle comprises a nucleic acid which is at least partially encapsulated within the polymer-based vehicle, wherein the nucleic acid either:
(a) encodes a protein which has a reduced abundance in a GTPase IMAP family member 5 (GIMAP5) deficient subject as compared to a healthy subject; or
(b) at least partially inhibits expression of a protein which has an increased abundance in a GIMAP5 deficient subject as compared to a healthy subject.

Embodiment 21 provides the composition of Embodiment 20, wherein the polymer-based vehicle comprises at least one selected from the group consisting of polyethyleneimine (PEI), poly-β-aminoester (PBAE), poly-L-lysine (PLL), chitosan, pullulan, dextran, and hyaluronic acid.

Embodiment 22 provides the composition of Embodiment 20 or 21, wherein the polymer-based vehicle is biodegradable.

Embodiment 23 provides the composition of any one of Embodiments 20-22, wherein the nucleic acid encodes a protein which has a reduced abundance in a GIMAP5 deficient subject as compared to a healthy subject.

Embodiment 24 provides the composition of Embodiment 23, wherein the protein which has a reduced abundance in a GIMAP5 deficient subject as compared to a healthy subject is an enzyme.

Embodiment 25 provides the composition of Embodiment 24, wherein the enzyme is GIMAP5.

Embodiment 26 provides the composition of Embodiment 25, wherein the nucleic acid comprises a messenger RNA (mRNA) which encodes a protein that shares at least 85%, 90%, 95%, 96%, 97%, 98%, or 99% sequence homology with SEQ ID NO:1.

Embodiment 27 provides the composition of Embodiment 26, wherein the mRNA encodes SEQ ID NO: 1.

Embodiment 28 provides the composition of Embodiment 23, wherein the protein which has a reduced abundance in a GIMAP5 deficient subject as compared to a healthy subject is selected from the group consisting of GATA4, MAF, and MEIS2.

Embodiment 29 provides the composition of Embodiment 28, wherein the nucleic acid comprises a mRNA which encodes a protein that shares at least 85%, 90%, 95%, 96%, 97%, 98%, or 99% sequence homology with a sequence selected from the group consisting of SEQ ID NO: 2, SEQ ID NO:3, and SEQ ID NO:4.

Embodiment 30 provides the composition of Embodiment 29, wherein the mRNA encodes a sequence selected from the group consisting of SEQ ID NO:2, SEQ ID NO:3, and SEQ ID NO:4.

Embodiment 31 provides the composition of any one of Embodiments 20-22, wherein the nucleic acid at least partially inhibits expression of a protein which has an increased abundance in a GIMAP5 deficient subject as compared to a healthy subject.

Embodiment 32 provides the composition of Embodiment 31, wherein the protein which has an increased abundance in a GIMAP5 deficient subject is selected from the group consisting of PDGFβ, VEGFα, APLN, MYC, and GATA6.

Embodiment 33 provides the composition of Embodiment 31 or 32, wherein the nucleic acid comprises a siRNA.

Embodiment 34 provides the composition of Embodiment 33, wherein the siRNA at least partially inhibits expression of a protein that shares at least 85%, 90%, 95%, 96%, 97%, 98%, or 99% sequence homology with a sequence selected from the group consisting of SEQ ID NO:5, SEQ ID NO:6, SEQ ID NO:7, SEQ ID NO:8, and SEQ ID NO:9.

Embodiment 35 provides the composition of Embodiment 33 or 34, wherein the siRNA at least partially inhibits expression of a protein with a sequence selected from the group consisting of SEQ ID NO:5, SEQ ID NO:6, SEQ ID NO:7, SEQ ID NO:8, and SEQ ID NO:9.

Embodiment 36 provides a recombinant viral vector, the vector comprising:
(a) an expression cassette comprising a nucleic acid sequence encoding a protein which has a reduced abundance in a GTPase IMAP family member 5 (GIMAP5) deficient subject as compared to a healthy subject; and
(b) an expression control sequence operably linked to the nucleic acid.

Embodiment 37 provides the recombinant viral vector of Embodiment 36, wherein the vector is an Adeno-associated virus (AAV) vector.

Embodiment 38 provides the recombinant viral vector of Embodiment 36 or 37, wherein the protein which has a reduced abundance in a GTPase IMAP family member 5 (GIMAP5) deficient subject as compared to a healthy subject is an enzyme.

Embodiment 39 provides the recombinant viral vector of Embodiment 38, wherein the enzyme is GIMAP5.

Embodiment 40 provides the recombinant viral vector of Embodiment 39, wherein the nucleic acid comprises a DNA sequence which encodes a protein that shares at least 85%, 90%, 95%, 96%, 97%, 98%, or 99% sequence homology with SEQ ID NO:1.

Embodiment 41 provides the recombinant viral vector of Embodiment 40, wherein the DNA sequence encodes SEQ ID NO:1.

Embodiment 42 provides the recombinant viral vector of Embodiment 36 or 37, wherein the protein which has a reduced abundance in a GTPase IMAP family member 5 (GIMAP5) deficient subject as compared to a healthy subject is GATA4.

Embodiment 43 provides the recombinant viral vector of Embodiment 42, wherein the nucleic acid comprises a DNA sequence which encodes a protein that shares at least 85%, 90%, 95%, 96%, 97%, 98%, or 99% sequence homology with SEQ ID NO:2.

Embodiment 44 provides the recombinant viral vector of Embodiment 43, wherein the DNA sequence encodes SEQ ID NO:2.

Embodiment 45 provides a pharmaceutical composition comprising the composition of any one of claims 1-35 or the recombinant viral vector of any one of claims 36-44 and a pharmaceutically acceptable carrier.

Embodiment 46 provides a method of treating, ameliorating and/or preventing liver disease and/or portal hypertension in a subject, the method comprising administering to the subject in need thereof a therapeutically effective amount of at least one selected from the group consisting of the composition of any one of claims 1-35, the recombinant viral vector of any one of claims 36-44, and the pharmaceutical composition of claim 45.

Embodiment 47 provides the method of Embodiment 46, wherein the subject is GIMAP5 deficient.

Embodiment 48 provides the method of Embodiment 46 or 47, wherein the subject has a loss-of-function (LOF) mutation in Gimap5.

Embodiment 49 provides the method of any one of Embodiments 46-48, wherein formation of a basement membrane in at least one liver endothelial cell of a subject is prevented, reduced, and/or reversed.

Embodiment 50 provides the method of any one of Embodiments 46-49, wherein loss of one or more fenestrations in at least one liver endothelial cell of a subject is prevented, reduced, and/or reversed.

Embodiment 51 provides the method of Embodiment 49 or 50, wherein the liver endothelial cell is selected from the group consisting of a liver sinusoidal endothelial cell (LSEC), liver macrovascular endothelial cell, or a liver lymphatic endothelial cell.

Embodiment 52 provides the method of any one of Embodiments 49-51, wherein the liver endothelial cell is a liver sinusoidal endothelial cell (LSEC).

Embodiment 53 provides the method of any one of Embodiments 46-52, wherein the subject is a mammal.

Embodiment 52 provides the method of Embodiment 53, wherein the mammal is a human.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A composition comprising one or more nucleic acid-lipid particles, wherein each nucleic acid-lipid particle comprises:
(a) a cationic lipid;
(b) a non-cationic lipid;
(c) a conjugated lipid that inhibits aggregation of two or more nucleic acid lipid particles; and
(d) a nucleic acid encapsulated within the nucleic acid-lipid particle, wherein the nucleic acid either:
(i) encodes a protein which has a reduced abundance in a GTPase IMAP family member 5 (GIMAP5) deficient subject as compared to a healthy subject; or
(ii) at least partially inhibits expression of a protein which has an increased abundance in a GIMAP5 deficient subject as compared to a healthy subject.

2. The composition of claim 1, wherein the cationic lipid comprises about 50 mol % to about 90 mol % of the total lipid present in the nucleic acid-lipid particle.

3. The composition of claim 1, wherein the non-cationic lipid is at least one selected from the group consisting of cholesterol and a phospholipid.

4. The composition of claim 1, wherein the non-cationic lipid comprises about 9.9 mol % to about 49.9 mol % of the total lipid present in the nucleic acid-lipid particle.

5. The composition of claim 1, wherein the conjugated lipid that inhibits aggregation of two or more nucleic acid-lipid particles comprises a polyethyleneglycol (PEG)-lipid conjugate.

6. The composition of claim 1, wherein the conjugated lipid comprises about 0.1 mol % to about 2 mol % of the total lipid present in the nucleic acid-lipid particle.

7. The composition of claim 1, wherein the nucleic acid encodes a protein which has a reduced abundance in a GIMAP5 deficient subject as compared to a healthy subject.

8. The method of claim 7, wherein the protein which has a reduced abundance in a GIMAP5 deficient subject as compared to a healthy subject is an enzyme.

9. The composition of claim 8, wherein the enzyme is GIMAP5.

10. The composition of claim 9, wherein the nucleic acid comprises a messenger RNA (mRNA) which encodes a protein that shares at least 85%, 90%, 95%, 96%, 97%, 98%, or 99% sequence homology with SEQ ID NO:1.

11. The composition of claim 10, wherein the mRNA encodes SEQ ID NO:1.

12. The composition of claim 7, wherein the protein which has a reduced abundance in a GIMAP5 deficient subject as compared to a healthy subject is selected from the group consisting of GATA4, MAF, and MEIS2.

13. The composition of claim 12, wherein the nucleic acid comprises a messenger RNA (mRNA) which encodes a protein that shares at least 85%, 90%, 95%, 96%, 97%, 98%, or 99% sequence homology with a sequence selected from the group consisting of SEQ ID NO:2, SEQ ID NO:3, and SEQ ID NO:4.

14. The composition of claim 13, wherein the mRNA encodes a sequence selected from the group consisting of SEQ ID NO:2, SEQ ID NO:3, and SEQ ID NO:4.

15. The composition of claim 1, wherein the nucleic acid at least partially inhibits expression of a protein which has an increased abundance in a GIMAP5 deficient subject as compared to a healthy subject.

16. The composition of claim 15, wherein the protein which has an increased abundance in a GIMAP5 deficient subject is selected from the group consisting of PDGFβ, VEGFα, APLN, MYC, and GATA6.

17. The composition of claim 15, wherein the nucleic acid comprises a small interfering RNA (siRNA).

18. The composition of claim 17, wherein the siRNA at least partially inhibits expression of a protein that shares at least 85%, 90%, 95%, 96%, 97%, 98%, or 99% sequence homology with a sequence selected from the group consisting of SEQ ID NO:5, SEQ ID NO:6, SEQ ID NO:7, SEQ ID NO:8, and SEQ ID NO:9.

19. The composition of claim 17, wherein the siRNA at least partially inhibits expression of a protein with a sequence selected from the group consisting of SEQ ID NO:5, SEQ ID NO:6, SEQ ID NO:7, SEQ ID NO:8, and SEQ ID NO:9.

20. A composition comprising one or more polymer-based vehicles, wherein the polymer-based vehicle comprises a nucleic acid which is at least partially encapsulated within the polymer-based vehicle, wherein the nucleic acid either:
(a) encodes a protein which has a reduced abundance in a GTPase IMAP family member 5 (GIMAP5) deficient subject as compared to a healthy subject; or
(b) at least partially inhibits expression of a protein which has an increased abundance in a GIMAP5 deficient subject as compared to a healthy subject.

21. The composition of claim 20, wherein the polymer-based vehicle comprises at least one selected from the group consisting of polyethyleneimine (PEI), poly-β-aminoester (PBAE), poly-L-lysine (PLL), chitosan, pullulan, dextran, and hyaluronic acid.

22. The composition of claim 20, wherein the polymer-based vehicle is biodegradable.

23. The composition of claim 20, wherein the nucleic acid encodes a protein which has a reduced abundance in a GIMAP5 deficient subject as compared to a healthy subject.

24. The composition of claim 23, wherein the protein which has a reduced abundance in a GIMAP5 deficient subject as compared to a healthy subject is an enzyme.

25. The composition of claim 24, wherein the enzyme is GIMAP5.

26. The composition of claim 25, wherein the nucleic acid comprises a messenger RNA (mRNA) which encodes a protein that shares at least 85%, 90%, 95%, 96%, 97%, 98%, or 99% sequence homology with SEQ ID NO:1.

27. The composition of claim 26, wherein the mRNA encodes SEQ ID NO:1.

28. The composition of claim 23, wherein the protein which has a reduced abundance in a GIMAP5 deficient subject as compared to a healthy subject is selected from the group consisting of GATA4, MAF, and MEIS2.

29. The composition of claim 28, wherein the nucleic acid comprises a mRNA which encodes a protein that shares at least 85%, 90%, 95%, 96%, 97%, 98%, or 99% sequence homology with a sequence selected from the group consisting of SEQ ID NO:2, SEQ ID NO:3, and SEQ ID NO:4.

30. The composition of claim 29, wherein the mRNA encodes a sequence selected from the group consisting of SEQ ID NO:2, SEQ ID NO:3, and SEQ ID NO:4.

31. The composition of claim 20, wherein the nucleic acid at least partially inhibits expression of a protein which has an increased abundance in a GIMAP5 deficient subject as compared to a healthy subject.

32. The composition of claim 31, wherein the protein which has an increased abundance in a GIMAP5 deficient subject is selected from the group consisting of PDGFβ, VEGFα, APLN, MYC, and GATA6.

33. The composition of claim 31, wherein the nucleic acid comprises a siRNA.

34. The composition of claim 33, wherein the siRNA at least partially inhibits expression of a protein that shares at least 85%, 90%, 95%, 96%, 97%, 98%, or 99% sequence homology with a sequence selected from the group consisting of SEQ ID NO:5, SEQ ID NO:6, SEQ ID NO:7, SEQ ID NO:8, and SEQ ID NO:9.

35. The composition of claim 33, wherein the siRNA at least partially inhibits expression of a protein with a sequence selected from the group consisting of SEQ ID NO:5, SEQ ID NO:6, SEQ ID NO:7, SEQ ID NO:8, and SEQ ID NO:9.

36. A recombinant viral vector, the vector comprising:
(a) an expression cassette comprising a nucleic acid sequence encoding a protein which has a reduced abundance in a GTPase IMAP family member 5 (GIMAP5) deficient subject as compared to a healthy subject; and
(b) an expression control sequence operably linked to the nucleic acid.

37. The recombinant viral vector of claim 36, wherein the vector is an Adeno-associated virus (AAV) vector.

38. The recombinant viral vector of claim 36, wherein the protein which has a reduced abundance in a GTPase IMAP family member 5 (GIMAP5) deficient subject as compared to a healthy subject is an enzyme.

39. The recombinant viral vector of claim 38, wherein the enzyme is GIMAP5.

40. The recombinant viral vector of claim 39, wherein the nucleic acid comprises a DNA sequence which encodes a protein that shares at least 85%, 90%, 95%, 96%, 97%, 98%, or 99% sequence homology with SEQ ID NO:1.

41. The recombinant viral vector of claim 40, wherein the DNA sequence encodes SEQ ID NO:1.

42. The recombinant viral vector of claim 36, wherein the protein which has a reduced abundance in a GTPase IMAP family member 5 (GIMAP5) deficient subject as compared to a healthy subject is selected from the group consisting of GATA4, MAF, and MEIS2.

43. The recombinant viral vector of claim 42, wherein the nucleic acid comprises a DNA sequence which encodes a protein that shares at least 85%, 90%, 95%, 96%, 97%, 98%, or 99% sequence homology with a sequence selected from the group consisting of SEQ ID NO: 2, SEQ ID NO:3, and SEQ ID NO:4.

44. The recombinant viral vector of claim 43, wherein the DNA sequence encodes a sequence selected from the group consisting of SEQ ID NO:2, SEQ ID NO:3, and SEQ ID NO: 4.

45. A pharmaceutical composition comprising the composition of claim 1 and a pharmaceutically acceptable carrier.

46. A method of treating, ameliorating and/or preventing liver disease and/or portal hypertension in a subject, the method comprising administering to the subject in need thereof a therapeutically effective amount of the composition of claim 1.

47. The method of claim 46, wherein the subject is GIMAP5 deficient.

48. The method of claim 46, wherein the subject has a loss-of-function (LOF) mutation in Gimap5.

49. The method of claim 46, wherein formation of a basement membrane in at least one liver endothelial cell of a subject is prevented, reduced, and/or reversed.

50. The method of claim 46, wherein loss of one or more fenestrations in at least one liver endothelial cell of a subject is prevented, reduced, and/or reversed.

51. The method of claim 49, wherein the liver endothelial cell is selected from the group consisting of a liver sinusoidal endothelial cell (LSEC), liver macrovascular endothelial cell, and a liver lymphatic endothelial cell.

52. The method of claim 49, wherein the liver endothelial cell is a liver sinusoidal endothelial cell (LSEC).

53. The method of claim 46, wherein the subject is a mammal.

54. The method of claim 53, wherein the mammal is a human.
